(19) United States
(12) Patent Application Publication
(10) Pub. No.: US 2017/0165200 A1
(43) Pub. Date: Jun. 15, 2017

(54) COMPOSITION OF LIPID-BASED NANOPARTICLES FOR SMALL MOLECULES AND MACROMOLECULES

(71) Applicant: University of North Texas Health Science Center, Fort Worth, TX (US)

(72) Inventors: Xiaowei DONG, Bedford, TX (US); Iok-Hou PANG, Grand Prairie, TX (US)

(73) Assignee: University of North Texas Health Science Center, Fort Worth, TX (US)

(21) Appl. No.: 15/355,318

(22) Filed: Nov. 18, 2016

Related U.S. Application Data
(60) Provisional application No. 62/258,030, filed on Nov. 20, 2015.

(51) Int. Cl. A61K 9/127; A61K 47/24; A61K 47/28; A61K 38/17; A61K 31/337; A61K 47/26; A61K 47/10; A61K 47/12; A61K 38/18; A61K 47/22; C12N 15/113

(52) U.S. Cl. CPC A61K 9/1275; A61K 47/22; A61K 47/24; A61K 47/28; A61K 38/1709; C12N 15/113; A61K 47/26; A61K 47/10; A61K 47/12; A61K 38/185; A61K 47/48315; A61K 31/337; C12N 2310/11; C12N 2320/32; C12N 2320/31; C12N 2310/14

(57) ABSTRACT

Described herein are nanoparticles comprising a mixture of a steroid, a phospholipid composition, an α-tocopheryl compound, and a therapeutic agent wherein the α-tocopheryl compound is presented on the surface of the nanoparticle. In some embodiments, the nanoparticles are useful for delivering a peptide or a protein. In some embodiments, the nanoparticles are formulated for ocular administration. In other embodiments, the nanoparticles are formulated to cross the blood brain barrier for the delivery of the therapeutic agents to the brain.

The present application claims benefit of priority to U.S. Provisional Application Ser. No. 62/258,030, filed Nov. 20, 2015, the entire contents of which are hereby incorporated by reference.

The invention was made with government support under Grant No. R03 NS087322-01 awarded by the National Institutes of Health. The government has certain rights in the invention.

### BACKGROUND

1. Field of the Disclosure

The present disclosure relates generally to the fields of medicine and pharmaceutics. In particular, it relates to nanoparticle compositions which target scavenger receptor class B type I (SR-BI) and deliver therapeutic agents to locations which express this receptor such as the brain, the eyes, and tumors.

2. Description of Related Art

Nerve Growth Factor (NGF) is one of the members of the neurotrophin family with multifaceted functions. It is well known for its role in survival, maintenance and differentiating actions on sympathetic and sensory neurons of peripheral nervous system and for the maintenance of functional integrity of cholinergic neurons in the central nervous system (CNS) (Aloe et al., 2012). Beneficial effects of NGF in various disease conditions, such as peripheral neuropathies, diabetes, skin ulcers, human immune deficiency virus, and ophthalmology, make it a potential therapeutic protein (Sofroniew et al., 2001). However, NGF administration through various routes like intravenous, subcutaneous or intra-cerebro ventricular (ICV) infusions caused a variety of undesirable and unwanted effects in patients (Apfel, 2002; McArthur et al., 2000; Eriksdotter Jonhagen et al., 1998). CERE-110, which was discontinued in Phase II clinical trial, is an adeno-associated viral vector that encodes the gene for NGF (Mandel, 2010). NsG0202 consists of an implantable encapsulated cell biodelivery device that secretes NGF (Wahlberg et al., 2012). However, both CRE-110 and NsG0202 are invasive requiring brain surgery procedures to incorporate them into the certain locations of the brain.

The use of proteins in medicine has been limited by their poor stability to proteolytic and hydrolytic degradation, low permeability across the barriers, and short biologic half-life in the circulatory system (Pinto Reis et al., 2006; Vaishya et al., 2015). Indeed, the half-life of NGF was about 5.4 min by intravenous injection in adult rats (Tria et al., 1994). Nanoparticles (NPs) are promising delivery systems for NGF. Thus, NPs could offer improved transport properties and pharmacokinetic profiles after systemic administration. Moreover, NPs could enhance biodistribution, modify release characteristics, reduce immunogenicity and target identified tissue with minimal distribution to normal tissues (Zhang et al., 2009). NPs have been studied to deliver NGF to cross the blood-brain barrier (BBB). NGF was absorbed on poly(butyl cyanoacrylate) (PBCA) NPs coated with polysorbate 80 (Kurakhmaeva et al., 2009). The results demonstrated that polysorbate-coated NGF PBCA NPs were able to cross the BBB and showed the anti-Parkinson effect in mice. Although about 96% of NGF was adsorbed onto the PBCA NPs after coating with polysorbate 80, the total NGF loading was low at only about 1.2 ng/mL leading to possible toxicity induced by the high dose of PBCA NPs needed. In other studies of the PBCA NPs, the competition of serum protein competed with polysorbate 80 as well as the rapid NP degradation in serum/plasma induced desorption of compounds adsorbed onto PBCA NPs within a few minutes (Olivier, 2005). Additionally, NGF has been directly incorporated into a liposome delivery system coated with RMP-7 to target to B2 receptor on brain microvascular endothelial cells. However, the entrapment efficiency (EE) of NGF was low at about 34% (Xie et al., 2005). NGF was also conjugated to an antibody of the transferrin receptor (Granholm et al., 1994) and the composition was able crossed the blood-brain barrier after peripheral injection. However, similar with the PBCA NPs, NGF was not protected by the formulations from degradation by enzymes. All of these compositions have significant issues which prevent them from becoming potential commercial applications for the delivery of NGF.

The studies showed that α-tocopherol originated from plasma is associated with HDLs and transported by scavenger receptor class B, type I (SR-BI) across the BBB (Balazs et al., 2004). Additional studies have demonstrated that HDL-associated α-tocopherol was taken up in 10-fold excess of HDL holoparticles, indicating efficiently selective uptake mediated by the SR-BI to cross the BBB (Goti et al., 2001). D-α-Tocopheryl polyethylene glycol succinate (vitamin E TPGS or simply TPGS) is a water soluble source of vitamin E with extended half-life and enhanced cellular uptake of the drug due to the combination of PEG and vitamin E (Structures shown in FIG. 1). Other nanoparticle compositions containing TPGS and other vitamin E derivatives have been developed by the inventors but these compositions do not present TPGS on the surface of the nanoparticle (Dong et al., 2009).

Similarly, delivery of therapeutic macromolecules, such as peptides, proteins, nucleic acids, polymers, or other large molecules, into the eye has been technically challenging. The anatomical structures of the eye limit entry of unaided foreign molecules into the intraocular space. As of now, other than intraocular injection, there is no clinically practical means to deliver therapeutically sufficient amount of macromolecules inside the eye. In the eye, SR-BI is also highly expressed at the corneal epithelial and endothelial cells, in the choroidal and scleral cells (Provost, 2003). The expression of this receptor facilitates selective uptake of high-density lipoprotein (HDL)-associated cholesteryl ester and α-tocopherol by receptor-mediated transport in the eyes. In addition, SR-BI has been shown to be overexpressed in many cancer cells. Therefore, the new compositions and methods for the drug delivery in this invention provide a novel, nonobvious, and useful approach to overcome the difficulties in drug delivery.

Therefore, new compositions and methods for the delivery of therapeutic agents which present α-tocopheryl compounds on the surface which are able to cross the blood brain barrier.

### SUMMARY

In some aspects, the present disclosure provides compositions which are formulated to target the scavenger receptor class B type I (SR-BI). In some embodiments, these compositions may be used to deliver therapeutic agents to any target which expresses the scavenger receptor class B type I including the brain, to a tumor, or to the eyes. In some embodiments, other targeted ligands may be coated on the surface of nanoparticle to target other receptors.

In some aspects, the present disclosure provides compositions comprising:
(a) a therapeutic agent;
(b) an α-tocopheryl compound;
(c) a phospholipid composition; and
(d) a steroid or steroid derivative,
   wherein the composition is formulated as a nanoparticle and the α-tocopheryl compound is substantially located on the surface of the nanoparticle.

In other aspects, the present disclosure provides compositions comprising:
(a) a therapeutic agent;
(b) an α-tocopheryl compound;
(c) a phospholipid composition;
(d) a steroid or steroid derivative, and
(e) an apolipoprotein;
   wherein the composition is formulated as a nanoparticle and the α-tocopheryl compound is substantially located on the surface of the nanoparticle.

In some embodiments, the therapeutic agent is a therapeutic protein. In some embodiments, the therapeutic protein is a growth factor. In some embodiments, the therapeutic protein is a neurotrophic factor. In other embodiments, the therapeutic protein is a neurotrophin such as nerve growth factor. In other embodiments, the neurotrophin is brain-derived neurotrophic factor, neurotrophin-3, or neurotrophin-4. In some embodiments, the neurotrophic factor is brain-derived neurotrophic factor, ciliary-derived neurotrophic factor, basic fibroblast growth factor, nerve growth factor, glial cell line-derived neurotrophic factor, neurotrophin-3, or neurotrophin-4. In some embodiments, the neurotrophic factor is brain-derived neurotrophic factor, ciliary-derived neurotrophic factor, or basic fibroblast growth factor.

In other embodiments, the therapeutic protein is an antibody. In other embodiments, the therapeutic protein is a mixture of antibodies. In some embodiments, the antibody is an anti-vascular endothelial growth factor (VEGF) antibody. In some embodiments, the antibody mixture reduces the biological activity of VEGF. In other embodiments, the therapeutic protein is a protein which binds VEGF. In some embodiments, the therapeutic protein is a mixture of proteins which binds VEGF. In some embodiments, the protein which binds VEGF reduces the biological activity of VEGF. In some embodiments, the proteins which bind VEGF reduce the biological activity of VEGF. In some embodiments, the protein binds placental growth factor (PIGF) and reduces the biological activity of PIGF. In some embodiments, the proteins bind PIGF and reduce the biological activity of PIGF. In some embodiments, the protein binds VEGF and PIGF and reduces the biological activity of both factors. In some embodiments, the proteins bind VEGF and PIGF and reduce the biological activity of both factors.

In some embodiments, the therapeutic protein is a mixture of a therapeutic protein and a polycationic protein molecule. In some embodiments, the polycationic protein molecule is polylysine, polyarginine, or protamine. In some embodiments, the polycationic protein molecule is protamine. In some embodiments, the therapeutic agent is a chemotherapeutic compound. In some embodiments, the therapeutic agent is a taxane such as docetaxel. In some embodiments, the therapeutic agent is a composition comprising a chemotherapeutic agent and a therapeutic oligonucleotide. In some embodiments, the therapeutic oligonucleotide is an antisense oligonucleotide. In some embodiments, the therapeutic oligonucleotide is an antisense oligonucleotide which reduces the expression of secretory clusterin (sCLU). In some embodiments, the therapeutic oligonucleotide is OGX-011.

In some embodiments, the α-tocopheryl compound is a pegylated derivative of α-tocopheryl. In some embodiments, the pegylated derivative of α-tocopheryl facilitates transportation of the composition across the blood-brain barrier. In some embodiments, the α-tocopheryl compound comprises a polyethylene glycol group with a molecular weight from about 100 g/mol to about 5000 g/mol. In some embodiments, the polyethylene glycol group has a molecular weight from about 500 g/mol to about 2500 g/mol. In some embodiments, the polyethylene glycol group has a molecular weight of about 1000 g/mol. In some embodiments, the polyethylene glycol group is linked to the α-tocopheryl compound by a linker group. In some embodiments, the linker group is a succinate group. In some embodiments, the α-tocopheryl compound is d-α-tocopheryl polyethylene glycol 1000 succinate.

In some embodiments, the phospholipid composition comprises two or more phospholipids. In some embodiments, the phospholipid composition comprises 2, 3, 4, 5, 6, 7, or 8 phospholipids. In some embodiments, the phospholipid composition comprises a mixture of phospholipids, triglycerides and apolipoprotein A-I which mimic a high density lipoprotein. In sonic embodiments, the phospholipid composition comprises a mixture of phospholipids and triglycerides. In some embodiments, the phospholipid composition comprises a first phospholipid of the formula:

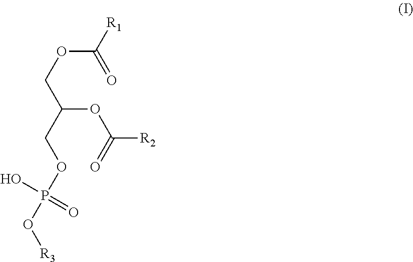

wherein:
R₁ and R₂ are each independently alkyl_{(C6-24)}, alkenyl_{(C6-24)}, or a substituted version of either of these groups; and
R₃ is hydrogen or —(CH₂)ₓRₐ, wherein:
   x is 1, 2, 3, 4, 5, or 6; and
   Rₐ is —NR′R″R′″⁺ or —CH(CO₂R_{b})NR_{c}R_{d}, wherein:
      R′, R″ and R′″ are each independently hydrogen, alkyl_{(C≦6)}, or substituted alkyl_{(C≦6)}; and
      R_{b}, R_{c}, and R_{d} are each independently hydrogen, alkyl_{(C≦6)}, or substituted alkyl_{(C≦6)};
         or a salt thereof.

In some embodiments, R₁ is alkyl_{(C6-24)}. In other embodiments, R₁ is alkenyl_{(C6-24)}. In some embodiments, R₂ is alkyl_{(C6-24)}. In other embodiments, R₂ is alkenyl_{(C6-24)}. In some embodiments, R₃ is —(CH₂)ₓRₐ, wherein:
x is 1, 2, 3, 4, 5, or 6; and
Rₐ is —NR′R″R′″+, wherein: R′, R″, and R′″ are each independently hydrogen, alkyl_{(C≦6)}, or substituted alkyl_{(C6≦6)}.

In some embodiments, x is 1, 2, 3, or 4. In some embodiments, x is 1, 2, or 3. In some embodiments, x is 1. In other embodiments, x is 2. In other embodiments, x is 3. In some embodiments, Rₐ is —NH₃⁺. In other embodiments, Rₐ is —N(CH₃)₃⁺. In some embodiments, Rₐ is —CH(CO₂R_{b})NR_{c}R_{d}, wherein:
R_{b}, R_{c}, and R_{d} are each independently hydrogen, alkyl_{(C≦6)}, or substituted alkyl_{(C≦6)},

In some embodiments, R_{b} is hydrogen. In some embodiments, R_{c} is hydrogen. In some embodiments, R_{d} is hydrogen. In some embodiments, R₃ is hydrogen. In some embodiments, the first phospholipid is further defined by the structure:

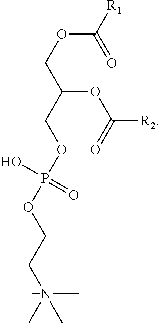

In other embodiments, the first phospholipid is further defined by the structure:

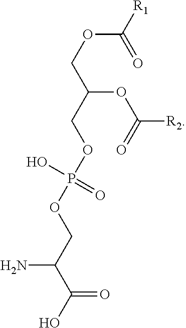

In some embodiments, the first phospholipid is a phosphatidylcholine.

In some embodiments, the phospholipid composition further comprises a second phospholipid. In some embodiments, the second phospholipid is further defined by formula I and wherein the second phospholipid is different from the first phospholipid. In some embodiments, the second phospholipid is further defined as:

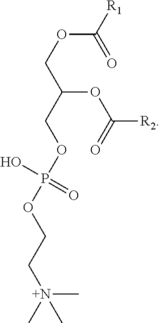

In other embodiments, the second phospholipid is further defined as:

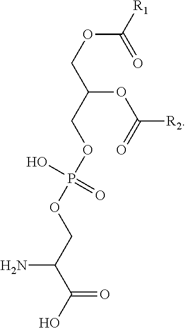

In some embodiments, R₁ is alkyl_{(C6-24)}. In other embodiments, R₁ is alkenyl_{(C6-24)}. In some embodiments, R₂ is alkyl_{(C6-24)}. In other embodiments, R₂ is alkenyl_{(C6-24)}. In some embodiments, the second phospholipid is a phosphatidylserine compound.

In some embodiments, the phospholipid composition further comprises a third phospholipid compound. In some embodiments, the third phospholipid compound is a compound of the formula:

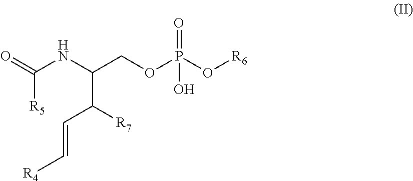

wherein:
R₄ and R₅ are each independently alkyl_{(C6-24)}, alkenyl_{(C6-24)}, or a substituted version of either of these groups; and
R₆ is hydrogen or —(CH₂)ₓRₐ, wherein:
   x is 1, 2, 3, 4, 5, or 6, and
   Rₐ is —NR′R″R′″⁺ or —CH(CO₂R_{b})NR_{c}R_{d}, wherein:
      R′, R″, and R′″ are each independently hydrogen, alkyl_{(C≦6)}, or substituted alkyl_{(C≦6)}; and
      R_{b}, R_{c}, and R_{d} are each independently hydrogen, alkyl_{(C≦6)}, or substituted alkyl_{(C≦6)};
R₇ is hydroxy or alkoxy_{(C≦6)}, acyloxy_{(C≦6)}, or a substituted version of either of these groups;
   or a salt thereof.

In some embodiments, R₄ is alkyl_{(C6-24)}. In other embodiments, R₄ is alkenyl_{(C6-24)}. In some embodiments, R₅ is alkyl_{(C6-24)}. In other embodiments, R₅ is alkenyl_{(C6-24)}. In some embodiments, R₆ is —(CH₂)ₓRₐ, wherein:
x is 1, 2, 3, 4, 5, or 6; and
Rₐ is —NR′R″R′″⁺, wherein: R′, R″, and R′″ are each independently hydrogen, alkyl_{(C≦6)}, or substituted alkyl_{(C≦6)}.

In some embodiments, x is 1, 2, 3, or 4. In some embodiments, x is 1, 2, or 3. In some embodiments, x is 1. In other embodiments, x is 2. In other embodiments, x is 3. In some embodiments, Rₐ is —NH₃⁺. In other embodiments, Rₐ is —N(CH₃)₃⁺. In some embodiments, R₇ is hydroxy. In other embodiments, R₇ is alkoxy_{(C≦6)}. In other embodiments, R₇ is acyloxy_{(C≦6)}. In some embodiments, the third phospholipid compound is further defined by the formula:

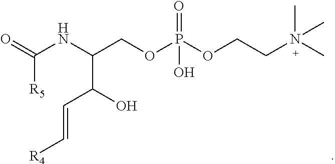

In some embodiments, the steroid or steroid derivative is a cholesterol ester_{(C≦24)}. In some embodiments, the steroid or steroid derivative is a cholesterol. In other embodiments, the steroid or steroid derivative is cholesterol oleate.

In some embodiments, the compositions further comprise an apoliprotein. In some embodiments, the apoliprotein is apolipoprotein A1. In other embodiments, the apoliprotein is a modified apolipoprotein A1. In some embodiments, the compositions further comprise a cell permeablizing agent. In some embodiments, the cell permeabilizing agent is a polyarginine peptide. In other embodiments, the cell permeabilzing agent is a pegylated polyarginine. In some embodiments, the compositions further comprise a targeting agent. In some embodiments, the targeting agent is an antibody, an antibody fragment, a peptide, a protein, a nucleic acid, or a small molecule. In some embodiments, the composition may further comprise an endosomal escaping agent, such as MGDG, diacylglycerol, a polyphosphoinositide or a fatty acid.

In some embodiments, the ratio of the phospholipid composition to the steroid or steroid derivative is from about 1:5 to about 15:1. In some embodiments, the ratio is 1:1 to about 10:1. In some embodiments, the ratio is from about 4:1 to about 8:1. In some embodiments, the ratio is about 4.9:1. In some embodiments, the ratio of the phospholipids in the phospholipid composition comprises a phosphatidylcholine to sphingomyelin ratio from about 10:1 to about 1:2. In some embodiments, the ratio is about 8:1 to about 2:1. In some embodiments, the ratio is about 5.2:1. In some embodiments, the ratio of the phospholipids in the phospholipid composition comprises a phosphatidylcholine to phosphotidylserine ratio from about 25:1 to about 1:1. In some embodiments, the ratio is from about 20:1 to about 10:1. In some embodiments, the ratio is about 15.7:1. In some embodiments, the steroid or steroid derivative comprises 0.5 w/w % to about 12.5 w/w % of the composition. In some embodiments, the steroid or steroid derivative comprises from about 2 w/w % to about 8 w/w %. In some embodiments, the steroid or steroid derivative comprises about 4.8 w/w %. In some embodiments, the phospholipid composition comprises from about 10 w/w % to about 45 w/w % of the composition. In some embodiments, the phospholipid composition comprises 15 w/w % to about 30 w/w %. In some embodiments, the phospholipid composition comprises about 23.6 w/w %. In some embodiments, the α-tocopheryl compound comprises from about 5 w/w % to about 60 w/w % of the composition. In some embodiments, the α-tocopheryl compound comprises from about 10 w/w % to about 50 w/w %. In some embodiments, the α-tocopheryl compound comprises about 14.8 w/w %.

In some embodiments, the therapeutic agent comprises from about 0.5 w/w % to about 25 w/w %. In some embodiments, the therapeutic agent comprises from about 1.0 w/w % to about 15 w/w %. In some embodiments, the therapeutic agent comprises about 3.2 w/w %. In other embodiments, the therapeutic agent comprises about 10 w/w %. In some embodiments, the compositions comprise the therapeutic agent and a polycationic molecule in a ratio from about 10:1 to about 1:10. In some embodiments, the ratio is from about 2:1 to about 1:2. In some embodiments, the ratio is about 1:1. In some embodiments, the lipoprotein comprises from about 20 w/w % to about 70 w/w % of the composition. In some embodiments, the lipoprotein comprises from about 40 w/w % to about 60 w/w %. In some embodiments, the lipoprotein comprises about 50.8 w/w %.

In some embodiments, the nanoparticle has a particle size from about 100 nm to about 500 nm. In some embodiments, the particle size is from about 100 nm to about 200 nm. In some embodiments, the particle size is from about 130 nm to about 170 nm. In other embodiments, the particle size is from about 200 nm to about 300 nm. In some embodiments, the particle size is from about 220 nm to about 270 nm. In some embodiments, the polydispersity index is less than 0.3. In some embodiments, the polydispersity index is less than 0.28.

In some embodiments, the compositions further comprise a pharmaceutically acceptable carrier. In some embodiments, the composition is formulated for administration: ocularly, ocular topically, intracamerally, subretinally, peribulbarly, retrobulbarly, orally, intraadiposaliy, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subchoroidally, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the compositions are formulated for administration via ocular topical administration, eye drop, or as an injection. In some embodiments, the compositions are formulated for ocular administration.

In another aspects, the present disclosure provides methods of preparing a therapeutic agent-loaded nanoparticle comprising:
(a) admixing a composition with an organic solvent and cholesterol, a composition with an organic solvent and a phospholipid composition, a composition with an organic solvent and an α-tocopheryl compound, and a composition with a solvent and a therapeutic agent to form a first reaction mixture;
(b) removing the organic solvent from the first reaction mixture to form a second reaction mixture;
(c) admixing the second reaction mixture to water by using a homogenizer or a sonication probe to form a prototype nanoparticle; and
(d) admixing one or more therapeutic agents with the prototype nanoparticle to form a therapeutic agent-loaded nanoparticle.

In yet another aspect, the present disclosure provides methods of preparing a therapeutic agent-loaded HDL mimicking nanoparticle comprising:
(a) admixing a composition with an organic solvent and cholesterol, a composition with an organic solvent and a phospholipid composition, and a composition with an organic solvent and an α-tocopheryl compound to form a first reaction mixture;
(b) removing the organic solvent from the first reaction mixture to form a second reaction mixture;
(c) admixing the second reaction mixture to water to form a prototype nanoparticle.
(d) admixing one or more therapeutic agents with the prototype nanoparticle to form a therapeutic agent-loaded nanoparticle; and
(e) admixing the therapeutic agent-loaded nanoparticle with apolipoprotein A-I to form a therapeutic agent-loaded HDL-mimicking nanoparticle.

In some embodiments, the methods further comprise homogenizing the prototype nanoparticle. In other embodiments, the methods further comprise homogenizing the therapeutic agent-loaded nanoparticle. In some embodiments, the organic solvent has a boiling point of less than 100° C. In some embodiments, the organic solvent is an alcohol_{(C≦8)} such as ethanol.

In some embodiments, the methods further comprise admixing one or more therapeutic agents, wherein admixing one or more therapeutic agents comprises:
(a) adding the therapeutic agent to the prototype nanoparticle;
(b) incubating the prototype nanoparticle and the therapeutic agent for a first time period at a first temperature; and
(c) stirring the prototype nanoparticle and the therapeutic agent for a second time period at a second temperature to form a therapeutic agent-loaded nanoparticle.

In some embodiments, the first time period is from about 1 minute to about 4 hours. In some embodiments, the first time period is from about 5 minutes to about 2 hours. In some embodiments, the first time period is about 30 minutes. In some embodiments, the first temperature is from about 25° C. to about 75° C. In some embodiments, the first temperature is from about 30° C. to about 50° C. In some embodiments, the first temperature is about 37° C.

In some embodiments, the second time period is from about 1 minute to about 4 hours. In some embodiments, the second time period is from about 5 minutes to about 2 hours. In some embodiments, the second time period is about 30 minutes. In some embodiments, the second temperature is from about 0° C. to about 37° C. In some embodiments, the second temperature is from about 15° C. to about 37° C. In some embodiments, the second temperature is about 25° C.

In some embodiments, the methods further comprise admixing a protein or peptide to the therapeutic agent-loaded HDL mimicking nanoparticle to form a protein and therapeutic agent-loaded HDL mimicking nanoparticle. In some embodiments, admixing the targeting agent comprises:
(a) adding the protein or peptide; and
(b) stirring the protein or peptide and the HDL mimicking nanoparticle for a third time period at a third temperature to obtain a protein and therapeutic agent-loaded HDL mimicking nanoparticle.

In some embodiments, the protein or peptide is an apolipoprotein. In some embodiments, the apolipoprotein is apolipoprotein A1. In some embodiments, the protein or peptide is a cell permabilizing agent. In some embodiments, the targeting agent is a R11 peptide comprising a PEG group.

In some embodiments, the third time period is from about 2 hours to about 24 hours. In some embodiments, the third time period is from about 6 hours to about 18 hours. In some embodiments, the third time period is about 12 hours. In some embodiments, the third temperature is from about 0° C. to about 37° C. In some embodiments, the third temperature is from about 15° C. to about 37° C. In some embodiments, the third temperature is about 25° C. In some embodiments, the homogenization comprises homogenizing the nanoparticle for a fourth time period from about 10 seconds to about 30 minutes. In some embodiments, the fourth time period is from about 30 seconds to about 10 minutes. In some embodiments, the fourth time period is about 5 minutes.

In still another aspect, the present disclosure provides compositions prepared according to the methods described herein.

In yet another aspect, the present disclosure provides methods of treating a disease or disorder in a patient comprising administering to the patient a therapeutically effective amount of a composition described herein.

In some embodiments, the disease or disorder is a central nervous system disorder. In some embodiments, the central nervous system disorder is Alzheimer's disease, Parkinson's disease, stroke, dementia, depression, schizophrenia, autism, Rett syndrome, anorexia nervosa, and bulimia nervosa. In other embodiments, the disease is an inflammatory disease. In some embodiments, the inflammatory disease is a rheumatic disease and multiple sclerosis. In other embodiments, the disease is a cardiovascular disease. In some embodiments, the cardiovascular disease is atherosclerosis, obesity, type 2 diabetes and metabolic syndrome. In other embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is prostate cancer such as a metastatic castration resistant prostate cancer. In other embodiments, the cancer is brain cancer.

In some embodiments, the methods further comprise administering a second anti-cancer therapy. In some embodiments, the second anti-cancer therapy is a second chemotherapeutic compound, radiotherapy, immunotherapy, or surgery.

In other embodiments, the disease or disorder is a disease or disorder of the eye. In some embodiments, the disease or disorder of the eye is glaucoma, age-related macular degeneration, diabetic retinopathy, retinal ischemic abnormalities, uveitis, endophthalmitis, or optic nerve trauma. In some embodiments, the disease or disorder is glaucoma.

In some embodiments, the patient is a mammal. In some embodiments, the patient is a human. In some embodiments, the composition is administered once. In other embodiments, the composition is administered two or more times.

In yet another aspect, the present disclosure provides methods of inducing neuronal growth comprising administering a composition described herein. In some embodiments, the composition is administered in vitro. In other embodiments, the composition is administered in vivo. In some embodiments, the composition is administered to a neuron.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims; the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present as a part of the α-tocopheryl compound.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The structure comparison of vitamin E TPGS and α-tocopheryl.

FIG. 2—Influence of homogenization times on NP preparation. Excipients were homogenized for 0, 1, 2, 3, 4, 5 or 6 min to form the NPs. Data are presented as the mean of particle size ±SD (n=3). #p>0.05.

FIG. 3—Relationship of Apo A-I loading and EE %. #p>0.05 for the EE %.

FIG. 4—Separation of the NGF NPs from free NGF by a gel filtration Separhose CL-4B column. The NGF NPs were measured based on particle intensity. Free NGF was measured by a Sandwich ELISA method.

FIG. 5—Long-term stability of batch 4-2 that did not contain Apo A-I. The batch was monitored for particle size and P.I. over 6 months. Data presented as mean particle size.

FIG. 6—Long-term stability of the prototype HDL-mimicking α-tocopheryl-coated NPs. Batch 2-4, 2-6, and 2-7 in Table 2B consist of three different compositions. Each batch was prepared in triplicate and monitored for particle size and P.I. over three months. For all tested NPs, P.I.<0.3. Data are presented as the mean particle size of three batches at the certain composition. #p>0.05. within the group.

FIG. 7—DTX NPs decreased IC₅₀ in DTX-resistant DU145 cells at 72 h (* p<0.05).

FIG. 8. Uptake of cy5-labeled miRNA363 NPs in prostate cancer cells by a confocal microscopy. The pictures show the imaging at the central section of cells analyzed by Z-stack image.

FIG. 9. Intercellular uptake of FITC-siRNA NPs in NCI/ADR-RES cells. The cells were treated with free FITC-siRNA and FITC-siRNA NPs for 3 hours. The Z-stack imaging was detected by a confocal microscope.

FIG. 10. Cellular uptake of Cy3-labeled anti-GAPDH siRNA NPs on PC3 cells (prostate cancer cells). The PC3 cells were treated with free siRNA or siRNA-loaded NPs for 4 hours at 37° C., which had an equivalent concentration of siRNA (6.20 μg/mL); The Z-stack imaging was taken by using a confocal microscope.

FIG. 11. Structure of MGDG (monogalactosyldiacyldiacylglycerol), a nonionic and non-bilayer lipid.

FIG. 12. Luciferase knockdown of anti-luciferase siRNA nanoparticles. In all of the treatments, the concentration of anti-luciferase siRNA was 12.3 pmole. Lipofectamine, a well-known commercial gene transfection agent, was used as a positive control. DOPE was mixed with PC to form nanoparticles for comparison with MGDG NPs. MGDG was incorporated with TPGS or PC in different concentrations to form different nanoparticles, so that the inventors were able to treat cells with different concentrations of MGDG (5 μm, 25 μm and 50 μm). (* p<0.05: significant difference compared to the control; #p>0.05: no significant difference compared to lipofectamine according to t-test),

FIGS. 13A-B. imaging of neurite outgrowth when the cells were treated with 50 ng/ml of free NGF (FIG. 13A) and NGF HDL-mimicking NPs (FIG. 13B).

FIG. 14. In vitro release profiles of free NGF and NGF NPs in 5% BSA-PBS solution (pH 7). Data are presented as the mean SD (n=4).

FIG. 15. Biodistribution of NGF NPs after mice were intravenously injected 40 μg/kg of NGF for 30 min (n=3). NGF NPs resulted in significantly higher NGF concentration in plasma compared to free NGF (p<0.05). For other tissues, NGF NPs led to lower NGF concentrations compared to free NGF (p<0.05).

### DESCRIPTION OF ILLUSTRATIVE EMBODIMENTS

In some aspects, the present disclosure provides nanoparticles which may be used to deliver therapeutic agents using nanoparticles which are coated with α-tocopheryl compounds. These compounds may be used to delivery compounds by crossing the blood retina barrier, blood brain barrier, to cancer cells, or to other tissues or cells that express scavenger receptor class B type I (SR-BI). In some embodiments, these nanoparticles present on their surfaces compounds or components which are recognized by scavenger receptor class B type I (SR-BI). These nanoparticles may be used with small molecule therapeutic agents, antibodies or functionalized antibodies, peptides, proteins, nucleic acids or functionalized nucleic acids, or other large molecule therapeutic agents. In some embodiments, these nanoparticles may be used to treat neurological disorders or ocular disorders.

### I. Chemical Definitions

When used in the context of a chemical group: “hydrogen” means —H; “hydroxy” or “hydroxyl” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ - - - - ” represents an optional bond, which if present is either single or double. The symbol “ [symbol] ” represents a single bond or a double bond. Thus, for example, the formula

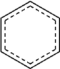

includes

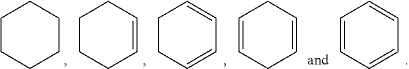

And it is understood that no one such ring atom forms part of more than one double bond. Furthermore, it is noted that the covalent bond symbol “—”, when connecting one or two stereogenic atoms, does not indicate any preferred stereochemistry. Instead, it cover all stereoisomers as well as mixtures thereof. The symbol “ [symbol] ”, when drawn perpendicularly across a bond (e.g.,

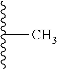

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

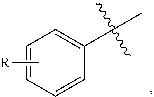

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

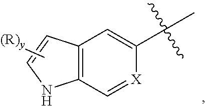

then R may replace any hydrogen atom attached to any of the ring atoms of either of the fused rings unless specified otherwise. Replaceable hydrogen atoms include depicted hydrogen atoms (e.g., the hydrogen atom attached to the nitrogen in the formula above), implied hydrogen atoms (e.g., a hydrogen atom of the formula above that is not shown but understood to be present), expressly defined hydrogen atoms, and optional hydrogen atoms whose presence depends on the identity of a ring atom (e.g., a hydrogen atom attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the number of carbon atoms in the group is as indicated as follows: “Cn” defines the exact number (n) of carbon atoms in the group/class. “C≦n” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. Compare with “alkoxy_{(C≦10)}”, which designates alkoxy groups having from 1 to 10 carbon atoms. Also compare “phosphine_{(C≦10)}”, which designates phosphine groups having from 0 to 10 carbon atoms. “Cn−n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefin_{C5}” are all synonymous.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂—(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “alkenyl” when used without the “substituted” modifier refers to an monovalent unsaturated aliphatic group with a carbon atom as the point of attachment, a linear or branched acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

A “sugar moiety” is a monovalent naturally or unnatural saccharide which is linked to the formula through a covalent bond between the formula and a heteroatom on the saccharide. Some non-limiting examples of carbohydrates which are included in the term sugar moiety include: glucose, galactose, xylose, ribose, arabinose, glyceraldehyde, erythrose, or mannose. The term may also include derivatized saccharides such as amino sugars or sulfosugars such as galactosamine, sialic acid, glucosamine, N-acetylglucosamine, or sulfoquinovose.

### II. NANOPARTICLE COMPOSITIONS AND FORMULATIONS

A. Hydrophobic Compounds

In some aspects of the present disclosure, the nanoparticle composition comprises a mixture of hydrophobic compounds such as phospholipids, steroids such as cholesterols, and other triglycerides. In some embodiments, these hydrophobic compounds are formulated to mimic the composition of a high density lipoprotein (HDL). In some embodiments, the nanoparticle comprises 1, 2, 3, 4, or more different types of hydrophobic compounds. Additionally, it is contemplated that the nanoparticle composition may comprise multiple different hydrophobic compounds within one type (e.g. multiple different phospholipids or different steroid derivatives). In some embodiments, the hydrophobic compound is a steroid or a steroid derivative. In other embodiments, the hydrophobic compound is a phospholipid or mixture of phospholipids. In some embodiments, the hydrophobic compound is a composition of two, three, or more phospholipids and one or more triglycerides. In other embodiments, the nanoparticle compositions comprise a steroid or a steroid derivative and a mixture of different types of phospholipids.

In some aspects, the nanoparticle composition comprises from about 0.5 w/w % to about 12.5 w/w % of a steroid or steroid derivative. The amount of steroid or steroid derivative may be from about 0.5 w/w %, 1 w/w %, 2 w/w %, 3 w/w %, 3.5 w/w %, 4 w/w %, 4.5 w/w %, 5 w/w %, 5.5 w/w %, 6 w/w %, 6.5 w/w %, 7 w/w %, 8 w/w %, 9 w/w %, 10 w/w %, 11 w/w %, 12 w/w %, to about 12.5 w/w %, or any range derivable therein. In some embodiments, the steroid or steroid derivative comprises about 4.8 w/w % of the nanoparticle composition.

In some aspects, the nanoparticle composition comprises from about 10 w/w % to about 45 w/w % of the phospholipid composition. The amount of phospholipid composition may be from about 10 w/w %, 12.5 w/w %, 15 w/w %, 17.5 w/w %, 20 w/w %, 21 w/w %, 22 w/w %, 22.5 w/w %, 23 w/w %, 24 w/w %, 25 w/w %, 27.5 w/w %, 30 w/w %, 32.5 w/w %, 35 w/w %, 37.5 w/w %, 40 w/w %, 42.5 w/w %, to about 45 w/w %, or any range derivable therein. In some embodiments, the phospholipid composition comprises about 4.8 w/w % of the nanoparticle composition.

1. Steroids and Steroid Derivatives

In some aspects of the present disclosure, the polymers are mixed with one or more steroid or a steroid derivative to create a nanoparticle composition. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

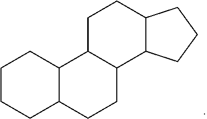 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

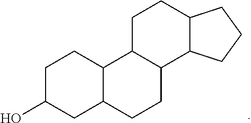 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

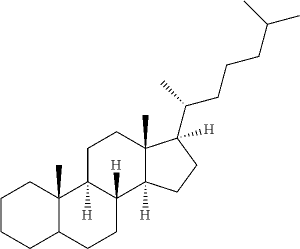

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

2. Phospholipids

In some aspects of the present disclosure, the polymers are mixed with one or more phospholipids to create a nanoparticle composition. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is further defined by a compound of the formula:

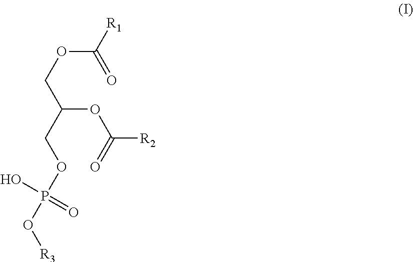

wherein:
R₁ and R₂ are each independently alkyl_{(C6-24)}, alkenyl_{(C6-24)}, or a substituted version of either of these groups; and
R₃ is hydrogen or —(CH₂)ₓRₐ, wherein:
   x is 1, 2, 3, 4, 5, or 6; and
   Rₐ is —NR′R″R′″⁺ or —CH(CO₂R_{b})NR_{c}R_{d}, wherein:
      R′, R″, and R′″ are each independently hydrogen, alkyl_{(C≦6)}, or substituted alkyl_{(C≦6)}; and
      R_{b}, R_{c}, and R_{d} are each independently hydrogen, alkyl_{(C≦6)}, or substituted alkyl_{(C≦6)}; or
         a compound of the formula:

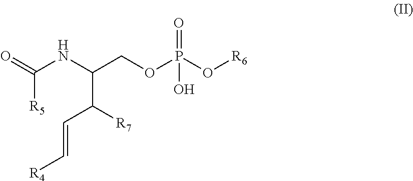

wherein:
R₄ and R₅ are each independently alkyl_{(C6-24)}, alkenyl_{(C6-24)}, or a substituted version of either of these groups; and
R₆ is hydrogen or —(CH₂)ₓRₐ, wherein:
   x is 1, 2, 3, 4, 5, or 6; and
   Rₐ is —NR′R″R′″⁺ or —CH(CO₂R_{b})NR_{c}R_{d}, wherein:
      R′, R″, and R′″ are each independently hydrogen, alkyl_{(C≦6)}, or substituted alkyl_{(C≦6)}; and
      R_{b}, R_{c}, and R_{d} are each independently hydrogen, alkyl_{(C≦6)}, or substituted alkyl_{(C≦6)};
R₇ is hydroxy or alkoxy_{(C≦6)}, acyloxy_{(C≦6)}, or a substituted version of either of these groups;
   or salts thereof.

In some embodiments, the phospholipid is a phosphatidylcholine. In other embodiments, the phospholipid is a phosphatidylserine. In other embodiments, the phospholipid is a sphingomyelin. In some embodiments, the nanoparticle composition comprises a mixture of phospholipids to obtain a phospholipid composition such as a mixture of phosphatidylserine, phosphatidylcholine, and sphingomyelin. In some embodiments, the phospholipid composition comprises a ratio of the first phospholipid to the second phospholipid from about 10:1 to about 1:2. In some embodiments; the ratio of the first phospholipid to the second phospholipid is from about 10:1, 9:1, 8:1, 7.5:1, 7:1, 6.5:1, 6:1, 5.5:1, 5:1, 4.5:1, 4:1, 3:1, 2:1, 1:1, to about 1:2, or any range derivable therein. In some embodiments; the ratio is about 5.2:1. In some embodiments, the nanoparticle composition comprises a third phospholipid. In some embodiments, the third phospholipid is present in a ratio to the first phospholipid from about 25:1 to about 1:1. In some embodiments, the ratio is from about 25:1, 24:1, 22:1, 20:1, 19:1, 18:1, 17:1, 16:1, 15:1, 14:1, 13:1, 12:1, 11:1, 10:1, 8:1, 6:1, 4:1, 2:1, to about 1:1, or any range derivable therein. In some embodiments, the ratio is about 15.7:1.

3. Triglycerides

In some aspects of the present disclosure, the nanoparticle compositions may further comprise one or more triglycerides. In various embodiments, the triglyceride is compound of the structure:

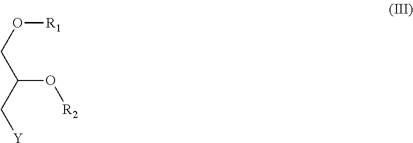

wherein:
R₁ and R₂ are each independently hydrogen or —C(O)—R₄; and
Y is hydrogen, hydroxy, a sugar moiety, or —OC(O)—R₄; wherein:
   R₄ is an alkyl_{(C1-25)}, alkenyl_{(C1-25)}, alkynyl_{(C1-25)}, or a substituted version of any of these groups; or a group of the formula: —C(O)—X—C(O)H, wherein X is an alkanediyl_{(C1-12)} or substituted alkanediyl_{(C1-12)};
provided that R₁, R₂, and Y are not all hydrogen.

In some embodiments, R₄ is selected from the group C₁-C₂₅ substituted or unsubstituted alkyl, C₁-C₂₅ substituted or unsubstituted alkenyl, C₁-C₂₅ substituted or unsubstituted alkynyl, and —C(O)—X—C(O)H, wherein X is —(CH₂)_{z}—, wherein Z=1-12. In some embodiments, R₄ is selected from the group C₁-C₂₅ alkyl, C₁-C₂₅ alkenyl, C₁-C₂₅ alkynyl, and —C(O)—X—C(O)H, wherein X is —(CH₂)_{z}—, wherein Z=1-12. In the above structure it is important to note that if one or more of R₁ and R₂ are —C(O)—R₄ and/or Y is —OC(O)—R₄, then a different R₄ group may be associated with R₁, R₂, and/or Y (e.g., R₁, R₂, and/or Y do not need to have the same R₄ group). In other embodiments, the Y group is a sugar moiety such as ether linked galactose, fructose, glucose, or xylose.

In some embodiments, R₁ or R₂ is —C(O)—R₄, wherein R₄ is C₄-C₁₈ alkyl, C₈-C₂₅ alkenyl, or C₈-C₂₅ alkynyl. In other embodiments, R₄ is —(CH₂)_{Y}—H, wherein Y=8-10. In some embodiments, R₁, R₂, and/or R₃ is a caprylic group, a capric group, a linoleic group, or a succinic group. In other embodiments, R₁ and R₂ are each independently an alkenyl group of 8-24 carbon atoms. In some aspects, the triglyceride is a composition comprising two or more different triglyceride molecules.

4. Cholesterol

In some aspects of the present disclosure, the nanoparticle compositions may further comprise a specific steroid class of steroids called cholesterol. Cholesterol has the formula:

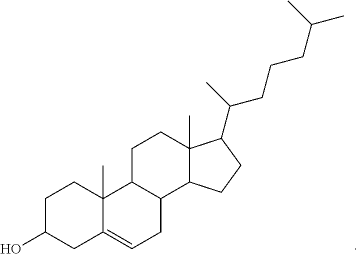 [SMILES: CC(C)CCCC(C)C1CCC2C3CC=C4CC(O)CCC4(C)C3CCC12C]

It is contemplated that any stereoisomers of the cholesterol molecule above. Furthermore, the molecule could be saturated such that the double bond in the B ring is hydrogenated to obtain a single bond. In other embodiments, the hydroxyl group in the A ring can also be oxidized to obtain a carbonyl. If the A ring has been oxidized, the carbonyl can also be an imino or thiocarbonyl group instead of an oxo group. In other embodiments, the cholesterol molecule is the natural isomer with the formula:

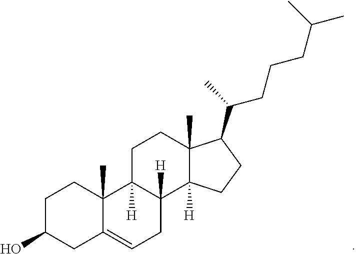

B. Vitamin E Components

In some embodiments, the nanoparticle composition comprises α-tocopherol or a derivative of α-tocopherol such as α-tocopheryl acetate or succinate. These compounds may also be conjugated with additional groups to add further functionalities. These additional groups include groups such as a hydrophobic group such as a fatty acid or long chain alkyl group on the free carboxyl group. In other embodiments, the nanoparticle composition is a PEGylated tocopheryl succinate compound. In some embodiments, the PEGylated tocopheryl succinate comprises a tocopherol succinate of a formula:

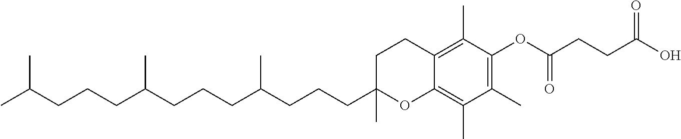 [SMILES: Cc1c(C)c2c(c(C)c1OC(=O)CCC(=O)O)CCC(C)(CCCC(C)CCCC(C)CCCC(C)C)O2]

and a PEGylated group attached to the free carboxyl group. In some embodiments, the PEG group comprises a repeating unit of ethylene glycol with a number of repeating units from 1 to 1,000. PEG is the polymeric form of ethylene glycol. The PEG portion of the compound has the formula:

Tocopheryl-(OCH₂CH₂)ₙOH (IV)

wherein the repeating unit, n, is an integer. The number of repeating units may be from about 1, 5, 10, 15, 20, 25, 50, 75, 100, 200, 300, 400, 500, 600, 700, 800, 900, to about 1,000 units, or any range derivable therein. In some aspects, the nomenclature used to describe PEG includes the average molecular weight of the polymer (e.g. PEG-800; PEG-1000, PEG-1200, etc.). As would be obvious to a person of skill in the art, the average molecular weight does not mean that any particular PEG component within the composition has the noted molecular weight but rather that the component as a whole has the average molecular weight corresponding to that value. In some embodiments, the PEG component can have a terminal hydrogen atom can be replaced with another group including but not limited to a C₁-C₆ alkyl group (e.g. a methyl group or an ethyl group), or a reactive moiety used to attach the PEG to another compound. For example, a PEG-1000 composition generally comprises PEG molecules with 16 and 17 repeating units as shown in the formula above, but may also comprises individual PEG molecules with less than 16 or more than 17 repeating units. As the value in the name of the PEG component represents the average molecular weight, the overall polymer average molecular weight may be modified to obtain an average molecular weight from less than 500 to over a 2500 g/mol (e.g. about 10 repeating units to about 40 repeating units). In some embodiments, the PEG component of the molecule has an average molecular weight equal to or less than PEG-1000.

In some aspects, the present disclosure provides nanoparticles which comprises from about 5 w/w % to about 60 w/w % of the α-tocopheryl compound. In some embodiments, the α-tocopheryl compound comprise from about 5 w/w %, 10 w/w %, 11 w/w %, 12 w/w %, 13 w/w %, 14 w/w %, 15 w/w %, 16 w/w %, 17 w/w %, 18 w/w %, 19 w/w %, 20 w/w %, 25 w/w %, 30 w/w %, 35 w/w %, 40 w/w %, 45 w/w %, 50 w/w %, 55 w/w %, to about 60 w/w %, or any range derivable therein.

C. Apolipoproteins

In some aspects, the nanoparticle compositions of the present disclosure may comprise one or more apolipoprotein. Apolipoproteins are associated with lipid metabolism and are present in a variety of different lipoproteins. These proteins are the major protein components of lipoproteins with Apolipoprotein A1 being the primary protein in high density lipoproteins. These proteins are associated with the transport of fat through the body. Without wishing to be bound by any theory, it is believed that these molecules bind to the outside of the lipid position of the lipoproteins to increase the lipoproteins'water solubility. Other apolipoprotein including apolipoprotein A-II, apolipoprotein A-IV, apolipoprotein A-V, apolipoprotein C such as apolipoproteins C-I, C-II, C-III, and C-IV, apolipoprotein D, apolipoprotein E, apolipoprotein H, and apolipoprotein L. In other embodiments, the apolipoprotein is apolipoprotein B such as apolipoprotein B48 or apolipoprotein B100. In some aspects, apolipoproteins A, C, and E share similar genetic origins and may be used in similar applications. It is also contemplated that one of these apolipoproteins may be modified such as through mutation or the attachment of a second compound or biologic component.

D. Therapeutic Agents

In some aspects, the nanoparticle compositions of the present disclosure comprise one or more therapeutic agents. In some embodiments, the nanoparticles comprise 1, 2, 3, 4, or 5 therapeutic agents. In some embodiments, the nanoparticles comprise 1 therapeutic agent or 2 therapeutic agents. In some aspects, the nanoparticle compositions comprise from about 0.5 w/w % to about 25 w/w %. In some embodiments, the nanoparticle compositions comprise from about 0.5 w/w %, 1 w/w %, 2 w/w %, 3 w/w %, 4 w/w %, 5 w/w %, 6 w/w %, 7 w/w %, 8 w/w %, 9 w/w %, 10 w/w %, 11 w/w %, 12 w/w %, 13 w/w %, 14 w/w %, 15 w/w %, 17.5 w/w %, 20 w/w %, 22.5 w/w %, to about 2.5 w/w %, or any range derivable therein. In some embodiments, the amount of therapeutic agent is about 3.8 w/w % of the composition. In other embodiments, the amount of the therapeutic agent is about 10 w/w % of the composition.

1. Nucleicids

In some aspects of the present disclosure, the nanoparticle compositions comprise one or more nucleic acids. In addition, it should be clear that the present disclosure is not limited to the specific nucleic acids disclosed herein. Formulations of pro-ISNP compositions may further comprise a nucleic acid based therapeutic agents. The present disclosure is not limited in scope to any particular source, sequence, or type of nucleic acid, however, as one of ordinary, skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). it is contemplated that the nucleic acid used in the present disclosure can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the nucleic acid is a complementary sequence to a naturally occurring sequence, or complementary to 75%, 80%, 85%, 90%, 95% and 100%.

In some aspects, the nucleic acid is a sequence which silences, is complimentary to, or replaces another sequence present in vivo. Sequences of 17 bases in length should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary, throughout their entire length and have no base mismatches. Other sequences with lower degrees of homology also are contemplated. For example, an anti sense construct which has limited regions of high homology, but also contains a non-homologous region (e.g., ribozyme; see below) could be designed. These molecules, though having less than 50% homology, would bind to target sequences under appropriate conditions.

Inhibitory RNA. As mentioned above, the present disclosure contemplates the use of one or more inhibitory nucleic acid for reducing expression and/or activation of a gene or gene product. Examples of an inhibitory nucleic acid include but are not limited to molecules targeted to an nucleic acid sequence, such as a microRNA, an siRNA (small interfering RNA), short hairpin RNA (shRNA), double-stranded RNA, an anti sense oligonucleotide, a ribozyme and molecules targeted to a gene or gene product such as an aptamer.

An inhibitory nucleic acid may inhibit the transcription of a gene or prevent the translation of the gene transcript in a cell. An inhibitory nucleic acid may be from 16 to 1000 nucleotides long, and in certain embodiments from 18 to 100 nucleotides long.

In some embodiment, an inhibitory nucleic acid is capable of decreasing the expression of a particular genetic product by at least 10%, at least 20%, at least 30%, or at least 40%, at least 50%, at least 60%, or at least 70%, at least 75%, at least 80%, at least 90%, at least 95% or more or any ranges in between the foregoing.

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties. In some embodiments, nucleosides of the present disclosure comprise one or more unmodified nucleobases. In certain embodiments, nucleosides of the present disclosure comprise one or more modified nucleobases.

2. Peptides and Proteins

The use of peptides and proteins as drugs continues to grow. As with many complex molecules, delivery issues may prevent the effective use of peptide/polypeptide drugs. Thus, the nanoparticles of the present disclosure may find use for the delivery of peptide/polypeptide drugs including but not limited to antibodies (Infliximab, Herceptin, Cetuximab, Rituximab), peptide hormones (insulin), clotting factors, anti-cancer peptides (Adalimumab, Aflibercept, Alemtuzumab, Bevacizumab, Bortezomib, Cilengitide, Triptorelin pamoate, Leuprolide acetate, Histrelin acetate, Goserelin acetate, Buserelin acetate, Abarelix acetate, Degarelix acetate), cytokines, interferons, interleukins IL-2, etc.), antivirals (Enfuvirtide), growth factors, enzymes (TPA), and a host of others (Teriparatide, Exenatide, Liraglutide, Lanreotide, Pramlintide, Ziconotide, Icatabant, Ecallantide, Tesamorelin, Mifamurtide and Nesiritude).

i. Therapeutic Antibodies

In some aspects, the nanoparticle compositions may further comprise an antibody or a fragment thereof that binds to at least a portion of an antigen are contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand.

Thus, by known means and as described herein, polyclonal or monoclonal antibodies, antibody fragments, and binding domains and CDRs (including engineered forms of any of the foregoing) may be created that are specific to the antigen, one or more of its respective epitopes, or conjugates of any of the foregoing, whether such antigens or epitopes are isolated from natural sources or are synthetic derivatives or variants of the natural compounds. Another variation is the construction of bispecific antibodies in which one heavy chain targeting one antigen and other heavy chain targeting a different antigen.

Examples of antibody fragments suitable for the present embodiments include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the V_{H} and V_{L} domains. Minibodies comprising a scFv joined to a CH₃ domain may also be made (Hu, et al., 1996).

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods. (Liu; et al., 2003).

ii. Protein Therapeutics

In some embodiments, the nanoparticle compositions may comprise or contain a therapeutic protein. The therapeutic protein may be a natural and nonnatural (e.g., recombinant) proteins, polypeptides, and peptides. The proteins may, by themselves, be incapable of passing (or which pass only a fraction of the administered dose) through the gastrointestinal mucosa or may be susceptible to chemical cleavage by acids or enzymes in the gastrointestinal tract or both. In addition to proteins, the nanoparticle composition also may include polysaccharides, and particularly mixtures of mucopolysaccharides, carbohydrates, lipids; other organic compounds.

Examples of proteins that may be comprised in a hydrogel copolymer of the present invention include, but are not limited to, synthetic, natural, or recombinant sources of: a growth hormone-releasing hormone, an interleukin (e.g., IL-1 beta); a growth factor (e.g., STEMGEN® (ancestim; stem cell factor); a basic fibroblast growth factor (e.g., high molecular weight FGF-2), a hepatocyte growth factor; erythropoietin (e.g., PROCRIT®, EPREX®, or EPOGEN® (epoetin-α); ARANESP® (darbepoetin-α); NEORECORMON®, EPOGIN® (epoetin-β); and the like); a blood factor (e.g., ACTIVASE® (alteplase) tissue plasminogen activator; NOVOSEVEN® (recombinant human factor VIIa); Factor VIIa; Factor VIII (e.g., KOGENATE®); Factor IX (e.g., BENEFIX®, RIXUBIS™, ALPROLIX™); hemoglobin; and the like); an antigen; a soluble receptor (e.g., a TNF-α-binding soluble receptor such as ENBREL® (etanercept); a soluble VEGF receptor; a soluble interleukin receptor; a soluble γ/δ T cell receptor; and the like); an enzyme (e.g., α-glucosidase; CERAZYME® (imiglucarase; β-glucocerebrosidase, CEREDASE® (alglucerase); an enzyme activator (e.g., tissue plasminogen activator); an angiogenic agent (e.g., vascular endothelial growth factor (VEGF); an anti-angiogenic agent (e.g., a soluble VEGF receptor); thrombopoietin; glial fibrillary acidic protein; a follicle stimulating hormone; a human alpha-1 antitrypsin; a leukemia inhibitory factor; a transforming growth factor; a tissue factor; a macrophage activating factor, a neutrophil chemotactic factor; fibrin; a leukemia inhibitory factor; or a protease inhibitor (e.g., β₂-macroglobulin). Combinations, analogs, fragments, mimetics or polyethylene glycol (PEG)-modified derivatives of these compounds, or other derivatives of any of the above-mentioned substances may also be suitable. Also suitable for use are fusion proteins comprising all or a portion of any of the foregoing proteins. One of ordinary skill in the art, with the benefit of the present disclosure, may recognize additional drugs, including drugs other than proteins, which may be useful in the compositions and methods of the present disclosure. Such drugs are still considered to be within the spirit of the present disclosure.

a. Growth Factors

In some embodiments, the present disclosure includes nanoparticle compositions which contain nerve growth factor (NGF) which may include any form of biologically active nerve growth factor including the β subunit of human nerve growth factor. The nerve growth factor may also include hybridized and modified forms of NGF which bind to the NGF receptor and retain NGF bioactivity. Modified forms of NGF may also include fusion proteins such as, for example, Iwai, et al., 1986 and Kanaya, et al., 1989, and NGF fragments and hybrids in which certain amino acids have been deleted or replaced while maintaining NGF bioactivity and receptor binding.

In some embodiments, the nanoparticle compositions with NGF contain human NGF (hNGF) including recombinant hNGF (rhNGF). Methods of preparing NGF are known in the art and include, for example, a baculovirus expression system (Barnett, et al., 1990), a yeast expression system (Kanaya, et al., 1989), a mammalian cell (CHO) expression system (Iwane, et al. 1990), a COS expression system (Bruce, et al., 1989), or bacterial expression system (Iwai, et al., 1986). The NGF which may be used herein includes NGF which is greater than 65% pure. In some embodiments, the NGF is greater than 85% pure. In some embodiments, the NGF is greater than 95% pure. In some embodiments, the NGF is greater than 98% pure. The purity may be determined by silver-stained SDS-PAGE or other means known to those skilled in the art.

In addition to NGF, other therapeutic agents include but not limited to pigmented epithelial derived factor (PEDF), basic fibroblast growth factor (bFGF), ciliary neurotrophic factor (CNTF). These therapeutic agents may be encapsulated into the nanoparticles, including those formulated for administration to the eyes. NGF, PEDF, bFGF, and CNTF have been demonstrated to be protective against various retinopathies in in vitro and in vivo models of ocular diseases, such as, glaucoma, age-related macular degeneration, diabetic retinopathy, retinal ischemic abnormalities, uveitis, optic nerve trauma, endophthalmitis and other ocular diseases.

3. Small Molecules

The overwhelming majority of drugs—antibiotics, antiviral, cancer chemotherapeutics, anti-hypertensives, statins, anti-depressives, and many others—and many others are categorized as “small molecules,” a general term applied to the class of compounds also described as organopharmaeuticals. In some aspects, these drugs or therapeutic agents are compounds which have a molecular weight of less than 2500 g/mol. In some embodiments, the therapeutic agents have a molecular weight from about 100, 150, 200, 250, 300, 350, 400, 450, 500, 550, 600, 650, 700, 750, 800, 900, 1000, 1250, 1500, 1750, 2000, 2250, to about 2500 g/mol. These therapeutic agents may be compounds which have a definitive structural formula and may be present as a neutral molecule or as a salt. In some embodiments; small molecule therapeutic agents are compounds which have a definitive chemical structure and formula which expressed through a specific connectivity of bonds and atoms. In another embodiment, the therapeutic agents used in the methods described herein are small molecule compounds which are not particular soluble in water. Some non-limiting examples of therapeutic agents are BCS classes II and IV compounds or other agents that similarly exhibit poor solubility. The BCS definition describes a compound in which the effective dosing is not soluble in 250 mL of water at a pH from 1-7.5. The USP categories “very slightly soluble” and “insoluble” describe a material that requires 1,000 or more parts of the aqueous liquid to dissolve 1 part solute. As used herein, when a compound is described as poorly soluble, it refers to a compound which has solubility in water of less than 1 mg/mL.

The methods of the present disclosure may be used to prepare nanoparticles using many classes of therapeutic agents including, but not limited to chemotherapeutics, agents for the prevention of restenosis, agents for treating renal disease; agents used for intermittent claudication, agents used in the treatment of hypotension and shock, angiotensin converting enzyme inhibitors; antianginal agents, anti-arrhythmics, anti-hypertensive agents, antiotensin ii receptor antagonists, antiplatelet drugs, β-blockers β1 selective, beta blocking agents, botanical products for cardiovascular indications, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators; potassium channel openers, anticonvulsants, antiemetics, antinauseants, anti-parkinson agents, antispasticity agents, cerebral stimulants, drugs to treat head trauma, drugs to assist with memory (e.g., to treat alzheimers/senility/dementia), drugs to treat migraine, drugs to treat movement disorders; also included are drugs to treat a disease such as multiple sclerosis, narcolepsy/sleep apnea, stroke, tardive dyskinesia; chronic graft versus host disease, eating disorders, learning disabilities, minimal brain dysfunction, obsessive compulsive disorder, panic, alcoholism, drug abuse, developmental disorders, diabetes; benign prostate disease, sexual dysfunction, rejection of transplanted organs, xerostomia, aids patients with kaposi's syndrome; antineoplastic hormones, biological response modifiers for cancer treatment; also included are vascular agents, cytoxic alkylating agents; cytoxic antimetabolics, cytoxics, immunomodulators, multi-drug resistance modulators, radiosensitizers, anorexigenic agents/CNS stimulants, antianxiety agents/anxiolytics, antidepressants, antipsychotics/schizophrenia, antimanics, sedatives and hypnotics, enkephalin analgesics, hallucinogenic agents, narcotic antagonists/agonists/analgesics, analgesics, epidural and intrathecal anesthetic agents, general, local, regional neuromuscular blocking agents sedatives, preanesthetic adrenal/acth, anabolic steroids, dopamine agonists, growth hormone and analogs, hyperglycemic agents, hypoglycemic agents, large volume parenterals (lvps), lipid-altering agents, nutrients/amino acids, nutritional lvps, obesity drugs (anorectics), somatostatin, thyroid agents, vasopressin, vitamins other than d, anti allergy nasal sprays, antiasthmatic dry powder inhalers, antiasthmatic metered dose inhalers, antiasthmatics (nonsteroidal), (antihistamines, antitussives, decongestants, etc.), beta-2 agonists, bronchoconstrictors, bronchodilators, cough-cold-allergy preparations, inhaled corticosteroids, mucolytic agents, pulmonary anti-inflammatory agents, pulmonary surfactants, anticholinergics, antidiarrheals, antiemetics, cathartics and laxatives, cholelitholytic agents, gastrointestinal motility modifying agents, h₂ receptor antagonists, inflammatory bowel disease agents, irritable bowel syndrome agents, liver agents, metal chelators, miscellaneous gastric secretory agents, miscellaneous gi drugs (including hemorrhoidal preparations), pancreatitis agents, pancreatic enzymes, prostaglandins, prostaglandins, gi, proton pump inhibitors, sclerosing agents, sucralfate, anti-progestins, contraceptives, oral contraceptives, estrogens, gonadotropins, gnrh agonists, gnrh antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors (including protamine sulfate & heparinase), blood drugs (e.g., drugs for hemoglobinopathies, hrombocytopenia, and peripheral vascular disease), prostaglandins, vitamin k, anti-androgens, androgens/testosterone, gnrh agonists, gnrh antagonists, aminoglycosides, antibacterial agents, sulfonamides, antibiotics, anti gonorrheal agents, anti-resistant antimicrobials, antisepsis immunomodulators, antitumor agents, cephalosporins, clindamycins, dermatologics, detergents, erythromycins, macrolides, anti-infectives (topical), other systemic antimicrobial drugs, otic-antibiotic in combination, penem antibiotics, penicillins, peptides antibiotic, sulfonamides, systemic antibiotics, immunomodulators, immunostimulatory agents, aminoglycosides, anthelmintic agents, antibacterial (bacterial vaginosis), antibacterial quinolones, antifungal (candidiasis), antifungal, systemic, anti-infectives/systemic, antimalarials, antimycobacterial, antiparasitic agents, antiprotozoal agents, antitrichomonads, antituberculosis, chronic fatigue syndrome, immunomodulators, immunostimulatory agents, macrolides, other drugs-aids related illnesses, other antiparasitic antimicrobial drugs, spiramycin, systemic antibiotics anti-gout drugs, corticosteroids, systemic, cyclooxygenase inhibitors, enzyme blockers, immunomodulators for rheumatic diseases, metalloproteinase inhibitors, nonsteroidal anti-inflammatory agents, non-steroidal anti-inflammatory agents, antifungals, antihistamines, contraceptives, detergents, non-narcotic analgesics, nsaids, vitamins, analgesics, nonnarcotic, antipyretics, counterirritants, muscle relaxant, anticaries preparations, antigingivitis agents, antiplaque agents, antifibrinolytics, chelating agents, alpha adrenergic agonists/blockers, antibiotics, antifungals, antiprotozoals, antivirals, beta adrenergic blockers, carbonic anhydrase inhibitors, corticosteroids, immune system regulators, mast cell inhibitors, nonsteroidal anti-inflammatory agents, prostaglandins, and proteolytic enzymes.

### III. FORMULATIONS AND THERAPEUTIC APPLICATIONS

A. Therapeutic Formulations

In some embodiments, the nanoparticles may be formulated as a pharmaceutical or therapeutic composition appropriate for the intended application. In certain embodiments, pharmaceutical compositions may comprise, for example, at least about 0.1% of the nanoparticle composition. In other embodiments, the nanoparticle composition may comprise between about 2% to about 75% of the weight of the unit, or between about 25% to about 60%, for example, and any range derivable therein.

The therapeutic compositions of the present embodiments are administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover; for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance; the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent may be taken every morning and/or every evening, regardless of when the subject has eaten or will eat.

The active compounds can be formulated for parenteral administration; e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and; the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The therapeutically compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic; oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Additionally, the pharmaceutical or therapeutic compositions may comprises one or more polycationic peptides or proteins such as protamine, polylysine, or polyarginine such that the therapeutic agent is formulated as a neutral salt or as a complex which contains significantly reduced charge.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion.

### IV. KITS

The present disclosure also provides kits. Any of the components disclosed herein may be combined in the form of a kit. In some embodiments, the kits comprise a nanoparticle composition as described above or in the claims.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the delivery components are combined in a single container. In other embodiments, some or all of the delivery components with the instant nanoparticle compositions are provided in separate containers.

The kits of the present disclosure also will typically include packaging for containing the various containers in close confinement for commercial sale. Such packaging may include cardboard or injection or blow molded plastic packaging into which the desired containers are retained or a glass vial containing a syringable composition. A kit may also include instructions for employing the kit components. Instructions may include variations that can be implemented.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many, changes can be made in the specific embodiments which are disclosed and still obtain a like or similar result without departing from the spirit and scope of the invention.

### Example 1

### Nerve Growth Factor Nanoparticles to Cross the Blood-Brain Barrier

The inventors aimed to develop novel HDL-mimicking α-tocopherol-coated nerve growth factor (NGF) nanoparticles targeting scavenger receptor class B type I (SR-BI) to cross the blood-brain barrier (BBB). Taguchi array was used to assist the NP development. Different ion-pair agents were employed to form an optimal ion-pair with NGF in order to facilitate the encapsulation of NGF. The novel HDL-mimicking α-tocopherol-coated NGF NPs were fully characterized in terms of particle size, entrapment efficiency and Apo A-I loading.

Materials and Cell Culture. Protamine from salmon, protamine grade X, protamine sodium salt USP, poly-lysine and cholesteryl Oleate (CO) were purchased from Sigma-Aldrich (St. Louis, Mo.). Sephadex G-50, Sephadex G-100, Sephacryl S-100 and Sepharose CL-4B were also purchased from Sigma-Aldrich (St. Louis, Mo.). PC, SM, and phosphatidylserine (PS) were purchased from Avanti polar lipids (Alabaster, Ala.). TPGS was provided by BSAF as a gift. Apo A-I was purchased from Athens research and technology (Athens, Ga.). Recombinant human NGF was purchased from Creative Biomart (Shirley, N.Y.). Neurite outgrowth staining kit was purchased from Molecular Probes by Life Technologies (Madison, Wis.). Bradford reagent was obtained from thermo scientific (Rockford, Ill.). Amicon ultra centrifugal filters-0.5 ml was obtained from Merk Millipore (Germany).

Optimization of preparation procedure for prototype HDL-mimicking NPs. Blank HDL-mimicking NPs were prepared by a self-assembly method. All excipients were dissolved in ethanol to prepare stock solutions. Certain amounts of PC (43.1%), SM (8.1%), PS (2.7%), CO (7.7%) and TPGS (38.4%) (percentages based on w/w) were added into a glass vial to form a thin film after removing ethanol by nitrogen. And then 1 ml of milliq water was added into the vial. Five different procedures were evaluated to hydrate the film to form NPs, including: 1) adding water at 50° C. and stifling at 50° C. for 30 min at 600 rpm, 2) adding water at 50° C. and stirring at room temperature (RT) for 30 min at 600 rpm, 3) adding water at RT and stirring at RT for 30 min at 600 rpm, and 4) adding water at 50° C. and homogenizing 5 min using a homogenizer at 8600 rpm, and 5) adding water at RT and homogenizing 5 min using a homogenizer at 8600 rpm. To further evaluate the influence of homogenization time on NP formation, the mixtures were homogenized for 0, 1, 2, 3, 4, 5, and 6 min after adding water at RT. After preparation, particle size and polydispersity index (P.I.) of NPs were measured using a Delsa Nano HC particle analyzer (Beckman Coulter, Calif.) at 90° light scattering at 25° C.

Development of Prototype HDL-Mimicking NPs by Taguchi Array

Taguchi array for NPs without Apo A-I. PC, SM and PS were selected as phospholipid components and CO was selected as the lipid component to develop the HDL-mimicking NPs. To simplify the design and quickly find the optimal compositions, the inventors considered phospholipids as one variable. The percentage of each phospholipid was fixed as PC (78%), SM (14%) and PS (3%) in the total phospholipids, which is close to the composition of phospholipids in natural HDLs. To evaluate different ratios of phospholipids and CO, the inventors designed two Taguchi arrays. In Taguchi array #1 (Table 2A and 2B), the ratio of phospholipids and CO was controlled around 1:1 (phospholipids/CO, w/w). Taguchi array for 3 levels 2 variables (phospholipids and CO) was used to give three different concentrations for each excipient. In Taguchi array #2 (Table 2C and 2D), an array for 2 levels 2 variables was used to give the ratio of phospholipids and CO around 4:1 to 8:1 (phospholipids/CO, w/w), NPs were prepared as described above. After forming the thin film, 1 ml of milliq water at RT was added into the vial and homogenized for 5 min to form NPs. To make TPGS-coated NPs, certain amounts of TPGS were added into Taguchi array (Table 2C and 2D) to give a total surfactant (phospholipids+TPGS) within 60 μg/ml to 110 μg/ml. Particle size and P.I. were measured as described above.
Tables 2A-2D. Taguchi array for development of HDL-mimicking α-tocopherol-coated NPs. Listed are the compositions per 1 ml NPs. A: Taguchi array with high contents of CO without TPGS, B: modified 2A by adding TPGS into the compositions, C: Taguchi array with low contents of CO without TPGS, and D: modified 2C by adding TPGS into the composition.

2A.

| Exper- | PC | SM | PS | CO | Particle size | |
|---|---|---|---|---|---|---|
| iment | (μg) | (μg) | (μg) | (μg) | (nm) | P.I. |
| 1-1 | 32 | 6 | 4 | 40 | 275.4 | 0.265 |
| 1-2 | 32 | 6 | 4 | 50 | 383.4 | 0.181 |
| 1-3 | 32 | 6 | 4 | 60 | 242.9 | 0.295 |
| 1-4 | 40 | 7.5 | 5 | 40 | 284.1 | 0.31 |
| 1-5 | 40 | 7.5 | 5 | 50 | 333.6 | 0.301 |
| 1-6 | 40 | 7.5 | 5 | 60 | 404.1 | 0.193 |
| 1-7 | 48 | 9 | 6 | 40 | 386.4 | 0.284 |
| 1-8 | 48 | 9 | 6 | 50 | 282.6 | 0.297 |
| 1-9 | 48 | 9 | 6 | 60 | 255.2 | 0.255 |

2B.

| Exper- | PC | SM | PS | CO | TPGS | Particle size | |
|---|---|---|---|---|---|---|---|
| iment | (μg) | (μg) | (μg) | (μg) | (μg) | (nm) | P.I. |
| 2-1 | 32 | 6 | 4 | 40 | 60 | 173 | 0.261 |
| 2-2 | 32 | 6 | 4 | 50 | 40 | 181.9 | 0.236 |
| 2-3 | 32 | 6 | 4 | 60 | 20 | 198.7 | 0.223 |
| 2-4 | 40 | 7.5 | 5 | 40 | 30 | 173.1 | 0.263 |
| 2-5 | 40 | 7.5 | 5 | 50 | 40 | 190 | 0.239 |
| 2-6 | 40 | 7.5 | 5 | 60 | 20 | 166 | 0.27 |
| 2-7 | 48 | 9 | 6 | 40 | 30 | 173.1 | 0.271 |
| 2-8 | 48 | 9 | 6 | 50 | 10 | 202 | 0.28 |
| 2-9 | 48 | 9 | 6 | 60 | 20 | 211.4 | 0.294 |

2C.

| Exper- | PC | SM | PS | CO | Particle size | |
|---|---|---|---|---|---|---|
| iment | (μg) | (μg) | (μg) | (μg) | (nm) | P.I. |
| 3-1 | 40 | 7.5 | 2.5 | 5 | 246.6 | 0.312 |
| 3-2 | 40 | 7.5 | 2.5 | 10 | 301.7 | 0.307 |
| 3-3 | 56 | 10.5 | 3.5 | 5 | 269.2 | 0.234 |
| 3-4 | 56 | 10.5 | 3.5 | 10 | 296.1 | 0.332 |

2D.

| Exper- | PC | SM | PS | CO | TPGS | Particle size | |
|---|---|---|---|---|---|---|---|
| iment | (μg) | (μg) | (μg) | (μg) | (μg) | (nm) | P.I. |
| 4-1 | 40 | 7.5 | 2.5 | 5 | 30 | 192.7 | 0.259 |
| 4-2 | 40 | 7.5 | 2.5 | 10 | 30 | 178.9 | 0.283 |
| 4-3 | 56 | 10.5 | 3.5 | 5 | 50 | 171.6 | 0.295 |
| 4-4 | 56 | 10.5 | 3.5 | 10 | 50 | 162 | 0.268 |

Optimization of loading Apo A-I in the prototype HDL-mimicking NPs. Based on the particle size and size distribution, the optimal compositions were selected to load Apo A-I, which are bolded in Tables 2B and 2D. To load Apo A-I on NPs, after homogenization for 5 min as described above, certain amounts of Apo A-I were added into each composition (Table 3). Different conditions were evaluated to load Apo A-I, including 2-hour stirring at RT, 4-hour stirring at RT, 4-hour stirring at RT followed with incubation at 4° C. overnight, and 4-hour stirring at RT followed with stirring at 4° C. overnight. Particle size and size distribution were measured as described above. Entrapment efficiency of Apo A-I was analyzed by ultrafiltration. Briefly, 0.2 ml of the NPs were added into Amicon Ultra (Molecular cutoff 100 KDa) and centrifuged at 14000 rpm at 4° C. for 3 min. After this, 400 μl water were added into the insert of Amicon to wash the membrane with the same centrifugation condition. Apo A-I was passed the membrane and washed with the same approach as described above to measure the recovery of Apo A-I in this separation method. The concentration of unloaded (free) Apo A-I in the filtrate was measured by Bradford assay. Loading and entrapment efficiency of Apo A-I were calculated as follows:

% loading=(drug added into NP)/(total weight of excipients)×100% Equation (1)

% entrapment efficiency=(1−unloaded drug/total drug added into NP)×100% Equation (2)

Furthermore, detailed studies on Apo A-I loadings were performed based on the composition of the batch 4-2. To optimize Apo A-I loading, different amounts of Apo A-I were added into the NPs (Table 4) by changing the amount of PC, but keeping the same amounts of SM, PS, CO and TPGS in the batch 4-2. Loading and entrapment efficiency of Apo A-I were measured and calculated as described above.

TABLE 3
Characterization of the prototype HDL-mimicking α-tocopherol-
coated NPs. Each batch (experiment) contained the
same composition as the corresponding batch in Table
2, except for the addition of Apo A-I.

| | | | | | Theoretical | |
|---|---|---|---|---|---|---|
| | Exper- | | | | loading | |
| | iment | Apo | Particle | | of Apo | EE % |
| Exper- | number in | A-I | size | | A-I | of Apo |
| iment | Table 2 | (μg) | (nm) | P.I. | (mole %) | A-I |
| 5-1 | 1-6 | 70 | 194.2 | 0.273 | 1.5 | 8 |
| 5-2 | 2-4 | 80 | 256.7 | 0.264 | 1.9 | 12 |
| 5-3 | 2-6 | 70 | 177.8 | 0.291 | 1.4 | 16 |
| 5-4 | 2-7 | 70 | 152 | 0.253 | 1.5 | 18 |
| 5-5 | 3-3 | 80 | 251.7 | 0.33 | 2.8 | 5 |
| 5-6 | 4-2 | 70 | 148.5 | 0.26 | 2.43 | 26 |
| 5-7 | 4-3 | 80 | 173.8 | 0.305 | 2.12 | 20 |

TABLE 4
Influence of Apo A-I loading on the prototype HDL-
mimicking α-tocopherol-coated NPs (n = 3).
Batch 5-6 in Table 3 was modified by changing the
contents of PC and Apo A-I to obtain batch 5-8 and 5-9.

| | | | | | Loading | |
|---|---|---|---|---|---|---|
| | | | | | of Apo | |
| | | Apo | Particle | | A-I | EE % |
| Exper- | PC | A-I | size | | (%, | of Apo |
| iment | (μg) | (μg) | (nm) | P.I. | w/w) | A-I |
| 5-6 | 40 | 70 | 145 ± 5 | 0.289 ± 0.012 | 43.8 | 31 ± 5.4 |
| 5-8 | 39 | 106 | 152 ± 5 | 0.265 ± 0.012 | 54.3 | 31 ± 3.6 |
| 5-9 | 38 | 140 | 156 ± 11 | 0.273 ± 0.001 | 61.4 | 26 ± 2.5 |

Particle size stability of prototype HDL-mimicking NPs at 4° C. The physical stability of the prototype HDL-mimicking NPs was assessed over time at 4° C. Prior to particle size measurement, nanoparticles were allowed to equilibrate to RT. One ml of NPs was used to measure the particle size and PI as described above.

Development of NGF-Loaded HDL-Mimicking NPs

Optimization of ion-pair complex for NGF. To efficiently load NGF into the NPs, poly-lysine and three types of protamines were tested to form an ion-pair complex with NGF. Protamines included protamine from salmon, protamine grade X and protamine sodium salt USP. Poly-lysine, protamines and NGF were dissolved in water at the concentration of 1 mg/ml. NGF was added into poly-lysine or protamine solutions at 0.8:1, 1:1, and 1:1.2 ratios (NGF:polymer, w/w). The complex was allowed to stand at RT for 10 min, and then diluted with 1 ml of water or PBS to measure particle size as described above and also to measure zeta potential using a Delsa Nano HC particle analyzer (Beckman Coulter, Calif.). The optimal ratio of the complex was determined according to particle size and zeta potential.

Preparation of NGF-loaded HDL-mimicking NPs. Poly-lysine and protamine USP were selected to prepare NU-loaded NPs. Briefly, 10 μg of NGF was mixed with 10 μg poly-lysine or protamine USP and kept for 10 min to form the complex. PC, SM, PS, CO and TPGS ethanol solutions (Table 6 below) were mixed and then ethanol was removed by nitrogen to form the thin film as described above. Two procedures were tested to add the NGF complex into NPs. In the first procedure, the NGF complex was added into the thin film, and then 1 ml of water at RT was added and homogenized for 5 min. In the second procedure, 1 ml of water at RT was first added into the thin film and homogenized for 5 min, and then the NGF complex was added into the solution. After addition of the NGF complex, the solution was incubated at 37° C. for 30 min, and then stirred at RT for 30 min. After cooling, the defined amount of Apo A-I was added into the solution and stirred at RT overnight to form the final NGF-loaded HDL-mimicking α-tocopherol-coated NPs. Particle size and zeta potential were measured as described above.

TABLE 6
The composition of the final HDL-mimicking
α-tocopherol-coated NGF NPs.

| | | | | | | Apo | Cationic | |
|---|---|---|---|---|---|---|---|---|
| Unit | PC | SM | PS | CO | TPGS | A-I | polymer | NGF |
| μg | 59 | 11 | 4 | 15 | 45 | 159 | 10 | 10 |
| w/w % | 18.8 | 3.6 | 1.2 | 4.8 | 14.4 | 50.8 | 3.2 | 3.2 |

Determination of NGF entrapment Efficiency in NGF-loaded HDL-mimicking NPs. Gel filtration chromatography was used to separate unloaded NGF from NGF NPs. To determine the fractions containing NGF, 200 μl of NGF solution (10 μg/ml) were added on a Sepharose 4B-CL column and eluted with PBS. Twelve fractions (about 1 ml for each) were collected and measured for the concentrations of NGF using a Sandwich ELISA method developed based on a Sandwich ELISA kit for NGF (R&D System, Minneapolis, Minn.). In a separate experiment, 200 μl of NGF HDL-mimicking NPs were eluted from the same column. The intensity in each fraction was measured using a Delsa Nano HC particle analyzer (Beckman Coulter, Calif.) to determine fractions containing NPs. The concentrations of NGF in fraction 5 to fraction 10 were measured and added together to calculate the amount of unloaded NGF. Loading and entrapment efficiency of NGF were calculated using equation (1) and (2) as described above.

Statistical analysis of the data including ANOVA and t-test, wherever needed, was conducted using Graph Pad Prism software. Results were considered significant if p<0.05.

Results

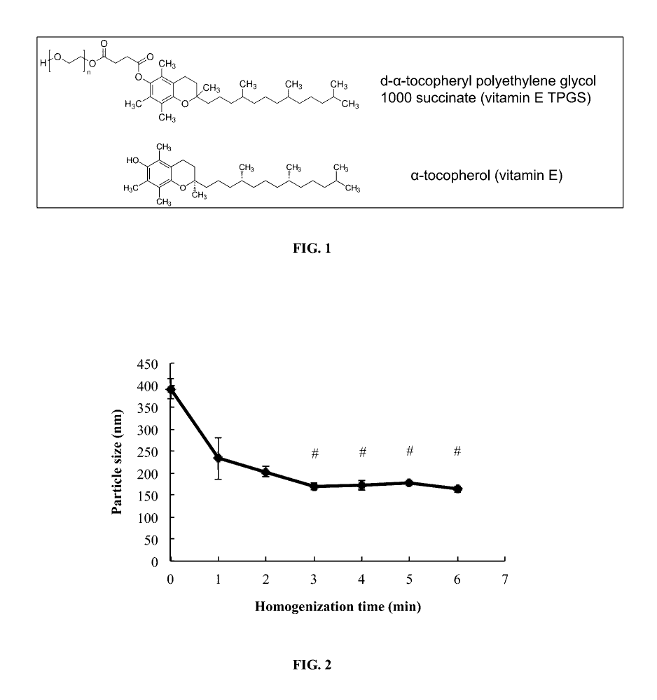

Optimal procedure for nanoparticle preparation. Significant efforts have been devoted to the use of recombinant lipoprotein-like NPs as drug delivery vehicles and diagnostic agents, because most of these particles resemble natural lipoprotein structures and are considered highly biocompatible and safe. Given the limitations of currently available preparation methods for scale-up of HDL-mimicking NPs, the inventors tested five different procedures to prepare the HDL-mimicking NPs by self-assembly. Table 1 below shows the results for four procedures and FIG. 2 shows the detailed study for the procedure using water at RT with 5-min homogenization. Efficient mixing is the key to prepare the NPs less than 200 nm. Increase of temperature did not help decrease of particle size. Since homogenization is a common technique used to prepare liquid formulations in industrial scales, the inventors enhanced the mixing efficiency by homogenization. With short-time homogenization, the inventors produced particle size at 183.9 nm with a narrow size distribution (P.I.<0.3). To further evaluate the influence of homogenization time on particle size, different homogenization time was studied. As shown in FIG. 2, there were no significant differences in particle size among 3-min 4-min, 5-min and 6-min homogenization (p>0.05). Thus, 5-min homogenization was selected to prepare NPs. The new preparation method developed here is easy to be scaled up with appropriate reproducibility.

TABLE 1
Evaluation of preparation procedures for blank
HDL-mimicking nanoparticle formation.

| | Particle | |
|---|---|---|
| Prepaation conditions | size (nm)^{a} | P.I.^{b} |
| 50° C. water + 30 min stirring at 50° C. | 347.7 ± 19.4 | 0.322 ± 0.0075 |
| 50° C. water + 30 min stirring at RT | 297.7 ± 21. 5 | 0.296 ± 0.0118 |
| RT water + 30 min stirring at RT | 335.4 ± 18.7 | 0.320 ± 0.0125 |
| 50° C. water + 5 min homogenization | 183.9 ± 7.0 | 0.276 ± 0.030 |

(^{a}The data are presented as the mean of the mean particle size of NPs in different batches ± SD (n = 3);
^{b}P.I. means polydispersity index that indicates size distribution of NPs. When P.I. < 0.35, NPs present as one single peak in the measurement (n = 3).)

Prototype HDL-mimicking NPs by Taguchi array. Accurate amounts of excipients in the NPs are keys to prepare self-assembled NPs. As mentioned above, natural HDLs are composed of multiple components. Experimental design based on a statistical method is desired to facilitate the finding of the accurate composition of the NPs formed by self-assembly. The inventors have used Taguchi array combined with simplex optimization to develop paclitaxel NPs in a previous study (Dong et at., 2009). Taguchi array effectively directed the nanoparticle development and optimization. Hence, the inventors chose Taguchi array to develop and optimize the HDL-mimicking NPs in this study. The detailed rationale to design the Taguchi array is described in the Method section. The results show in Tables 2A and 2B. Without TPGS, particle size was >250 nm (Tables 2A and 2C). The addition of TPGS decreased particle size (<200 nm) and also narrowed size distribution (Tables 2B and 2D), but further increasing TPGS did not influence particle size as compared the batch 2-1 to other batches. The ratio of phospholipids and CO did not influence to particle size as small particle size (<200 nm) was obtained in both Taguchi arrays (Tables 2B and 2D) As shown in Table 2B, batch 2-1, 2-4, 2-6 and 2-7 gave smaller particle size compared to other batches. However, the total amount of the surfactants in batch 2-1 was very high, potentially leading to instability of NPs; thus, batch 2-4, 2-6 and 2-7 were selected to load Apo A-I. In Table 2D, all four batches produced similar NPs. The inventors chose batch 4-2 and 4-3 to represent batches with different amounts of CO to load Apo A-I.

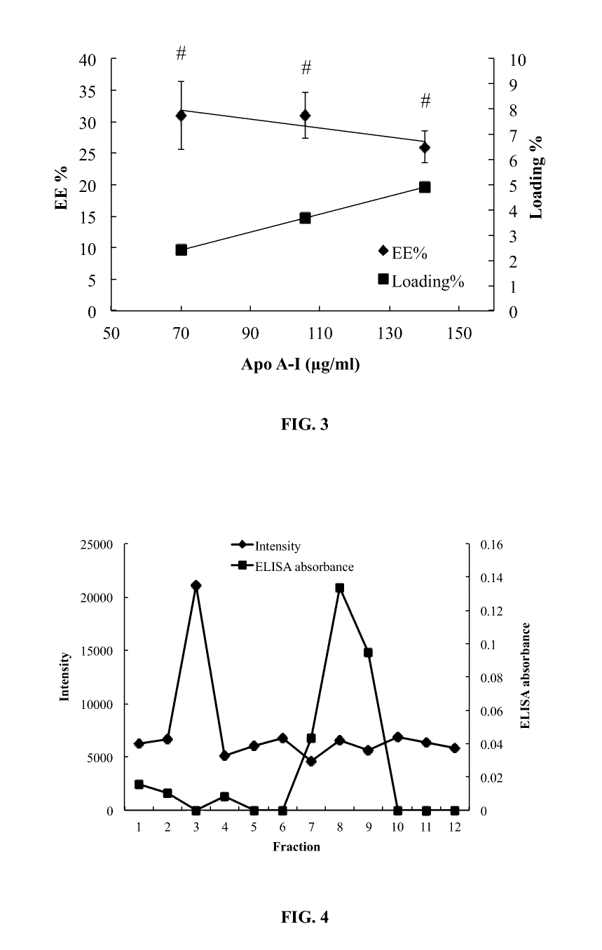

Apo A-I entrapment efficiency. The inventors used membrane separation to measure EE % of Apo A-I. Proteins have trend to bind with separation membranes. Thus, the inventors measured the recovery of Apo A-I from Amicon Ultra. The result showed that about 50% Apo A-I were detected in the filtrate after the initial centrifugation. After the inventors used 400 μl of water to wash the membrane, the recovery of Apo A-I in the filtrate was 84.3%±4.5, demonstrating that the method was sufficient to collect free Apo A-I in the filtrate. The inventors loaded Apo A-I into the batches highlighted in Table 2B and 2D in order to prepare HDL-mimicking NPs. Different conditions were tested to load Apo A-I into the NPs. The results showed that the initial 4-hour stirring at RT was crucial to get homogenous NPs, and incubation overnight was important to get appropriate EE % of Apo A-I. Thus, the inventors selected 4-hour stirring at RT followed with incubation at 4° C. overnight to load Apo A-I. It was observed that drug formulations with TPGS resulted in high drug encapsulation efficiency along with high cellular uptake and therapeutic effects in in vitro and in vivo respectively (Zhang et al., 2012). To understand the influence of TPGS on EE % of Apo A-I, the inventors also selected batch 1-6 from Table 2A and batch 3-3 from Table 2C as representative batches to load. Apo A-I. As shown in Table 3, all of the batches (batch 5-2, 5-3, 5-4, 5-6, and 5-7) that contained TPGS in the compositions had higher EE % of Apo A-I, compared to the batches without TPGS (batch 5-1 and 5-5). These results suggested that addition of TPGS improved EE % of Apo A-I. The highest EE % of Apo A-I was provided by the batch 5-6. To clearly know the influence of Apo A-I loading on its EE %, the inventors designed another two batches by only replacing the amount of PC with Apo A-I while keeping the same amounts of other excipients in the batch 5-6 (Table 4). By this design, the inventors minimized the influence from the change of NP composition. The profiles show that increasing Apo A-I loading did not change EE % of Apo A-I (FIG. 3). The EE % of Apo A-I was over 26%—about 3-fold higher than those reported in literatures. Consequently, the real content of Apo A-I in the NPs were over 16% close to the Apo A-I content in natural HDLs. The inventors chose the composition of the batch 5-8 to prepare NGF-loaded NPs. In the preparation, the inventors added about 0.14 mg/ml of Apo A-I to achieve a sufficient Apo A-I content in the NPs, which dramatically decreased the use of Apo A-I compared to previously reported NPs.

Ion-pair complex for NGF. NGF is a 120-amino acid polypeptide homodimer. It presents as monomer with 13 KD and forms dimer by a disulfide bond in aqueous condition. Positively charged amino acids are dominate in the NGF monomer chain; however, after folding, the surface potential of the NGF dimer is negative as positively charged basic groups forms a positive groove at one end of the dimer that responsible for the binding affinity of NGF to its receptor. Therefore, the inventors hypothesized that a cationic polymer would be a suitable complex agent to form an ion-pair complex with NGF to facilitate encapsulation of NGF into the NPs. First, the inventors tested if cationic polymers could form complexes with NGF. After mixing protamine with NGF at 1:1 ratio (w/w), the inventors easily visualized formation of white precipitates, directly indicating the formation of the complex. Next, the inventors measured particle size and zeta potential of the complexes that were formed by mixing each cationic polymer with NGF at different ratios. As expected, zeta potential changed from positive to negative while decreasing the concentrations of protamine, protamine sulfate USP and poly-D-lysine (Table 5 below). The results confirm that NGF has negative charge on the surface and using cationic polymers for complexation is appropriate for NGF. PC and SM are neutral phospholipids and TPGS is a non-ionic surfactant. PS is negative-charged phospholipids. Thus, the whole NPs are negatively charged. A desirable complex should not only contain a minimal amount of the cationic polymer to produce sufficient complexation but also keep the complex slightly positively charged to be entrapped into the negatively charged HDL-mimicking NPs. As shown in Table 5, large aggregation was shown at the ratio of 1:1 of NGF to protamine, suggesting that a complex formed and tended to aggregate. Importantly, at the ratio of 1:1, the complex had slightly positive charge, which was preferred as described above. Compared with other tested protamines, protamine sulfate USP showed more favorite properties in terms of particle size and zeta potential. Moreover, protamine sulfate USP is approved by the Food and Drug Administration for injection. Therefore, the inventors chose protamine sulfate USP as the ion-pair agent to prepare NGF HDL-mimicking NPs. In contrast to protamine, the inventors did not observe the same trend on particle size for poly-D-lysine. The ratio of NGF to poly-D-lysine at 1:1 and 1.2:1 produced similar particle size, but the zeta potential was more sensitive for the change compared to protamines. These results suggested that protamines were superior to poly-D-lysine for NGF as the complexation using poly-D-lysine could more difficult to be qualified and controlled than those using protamines. The inventors included poly-D-lysine in the following study as a comparison. One concern while using charge-charge interaction for formulations is instability of the ion-pair complex because of the competition from other ions in physiological fluid. To verify the stability of the NGF complexes, the inventors mixed the NGF/protamine USP or NGF/poly-D-lysine complexes with PBS and then measured particle size. In PBS, particle size of the NGF/protamine complex and the NGF/poly-D-lysine complex was 725.3 nm and 957.6 nm, respectively, indicating both complexes were stable.

TABLE 5
Ion-pair complexes of protamines or poly-D-Lysine with NGF at different ratios.

| | Protamine | | Protamine | | Protamine | | Poly- | |
|---|---|---|---|---|---|---|---|---|
| | free base | | salt from salmon | | sulfate USP | | D-Lysine | |
| NGF:Polycation | Size | Potential | Size | Potential | Size | Potential | Size | Potential |
| (w/w) in water | (nm) | (mV) | (nm) | (mV) | (nm) | (mV) | (nm) | (mV) |
| 0.8:1 | 554.3 | 12.22 | 278.5 | 0.34 | 562.6 | 0.86 | 529.3 | 0.54 |
| 1:1 | 863.6 | 0.58 | 589.4 | 0.22 | 802.6 | 0.30 | 805.5 | −0.82 |
| 1.2:1 | 596.4 | −0.71 | 543.7 | 0.59 | 356.0 | −0.32 | 830.0 | −4.53 |

NGF-loaded HDL-mimicking NPs. To load 10 μg/ml of NGF, the inventors modified the composition of the batch 5-8 (Table 4) by increasing each excipient for 1.5 times. The final composition of the NGF HDL-mimicking NPs is shown in Table 6. The NGF loading was 3.2% and the Apo A-I loading was 50.8%. Two procedures to add the NGF complex into the NPs were evaluated. In both procedures, adding NGF complex before and after homogenization, did not show difference on particle size and size distribution. To protect the bioactivity of NGF after nanoparticle preparation, the inventors decided to add NGF complex after homogenization. Also, after addition of Apo A-I, stirring the NPs at RT overnight provided higher EE % of NGF compared to incubation at 4° C. overnight. As a consequence, NGF HDL-mimicking NPs were prepared by stirring at RT overnight after adding Apo A-I.

To measure the EE % of NU, the inventors first tried to use Amicon Ultra (molecule cutoff 100 kDa) to separate free NGF and NGF-loaded NPs. However, free NGF did not pass the membrane, probably due to the formation of the high molecular weight of the NGF dimer (26 KDa) in aqueous solution. The inventors next tested several gel filtration column including Sephadex G-50, Sephadex G-100, Sephacryl S-100 and Sepharose CL-4B. Finally, Separhose CL-4B completely separated NGF NPs and free NGF. As shown in FIG. 4, fractions of 2 to 4 contained NGF NPs. The inventors calculated the EE % of NGF based on the concentrations of free NGF from fraction 6 to fraction 10 after the column separation. Different ELISA methods were evaluated to quantitatively measure the concentration of NGF. A direct ELISA method worked very well for NGF standard solution that was in PBS. However, cationic polymers, protamine sulfate USP and poly-D-lysine, increased the NGF absorbance in the direct ELISA method. Next, the inventors evaluate a commercial NGF ELISA kit. Protamine sulfate USP and poly-D-lysine did not interfere with NGF measurement using the sandwich ELISA method. Characterization of NGF HDL-mimicking NPs is shown in Table 7 below. Both NGF HDL-mimicking NPs had relatively narrow size distribution. D90, the size which 90% of the distribution lies below, was smaller than 550 nm and D10, the size which 10% of the distribution lies below, was bigger than 75 nm. As expected, NGF/protamine sulfate USP NPs had higher NGF EE % than NGF/poly-D-lysine NPs. The variation of zeta potential on NGF/protamine sulfate USP NPs also was smaller than that of NGF/poly-D-lysine NPs. It could be because the charge density of poly-p-lysine is relatively high compared to protamine sulfate USP; thus, small change on poly-D-lysine amounts significantly influenced complex formation and zeta potential. Also, the NGF/poly-D-lysine complex had negative zeta potential that may not prefer the negatively charged NPs. The final NGF NPs had negative zeta potential that is favorable for cell uptake and nanoparticle stability. Hazardess organic solvents (e.g. chloroform) were not used, and all excipients in the NPs are naturally present, minimizing the toxicity of the NPs. In this study, PBS was used to wash the gel filtration column to separate free NGF and NGF NPs for measurement of the EE %. Unloaded NGF and loosely bound NGF (on the nanoparticle surface) were separated and washed out as free NGF from fraction 6 to 10. Therefore, the 65.9% of NGF measured for the EE % should be entrapped in the core of the NPs so that they did not dissociate from the NPs during the column separation and elution by PBS. This suggests that the HDL-mimicking α-tocopherol-coated NPs could protect NGF from degradation and systemically deliver NGF to treat diseases.

TABLE 7
Characterization of the HDL-mimicking α-tocopherol-
coated NGF NPs using protamine sulfate USP and Poly-
D-Lysine as ion-pair agents, respectively (n = 3).

| NGF HDL- | Particle | | EE % | Zeta poten- |
|---|---|---|---|---|
| mimicking NPs | size (nm) | P.I. | of NGF | tial (mV) |
| Protamine | 171.4 ± 5 | 0.289 ± 0.012 | 65.9 ± 1.4 | −12.5 ± 1.9 |
| sulfate USP | | | | |
| Poly-D-lysine | 152 ± 5 | 0.265 ± 0.012 | 49.1 ± 1.7 | −24.9 ± 8.1 |

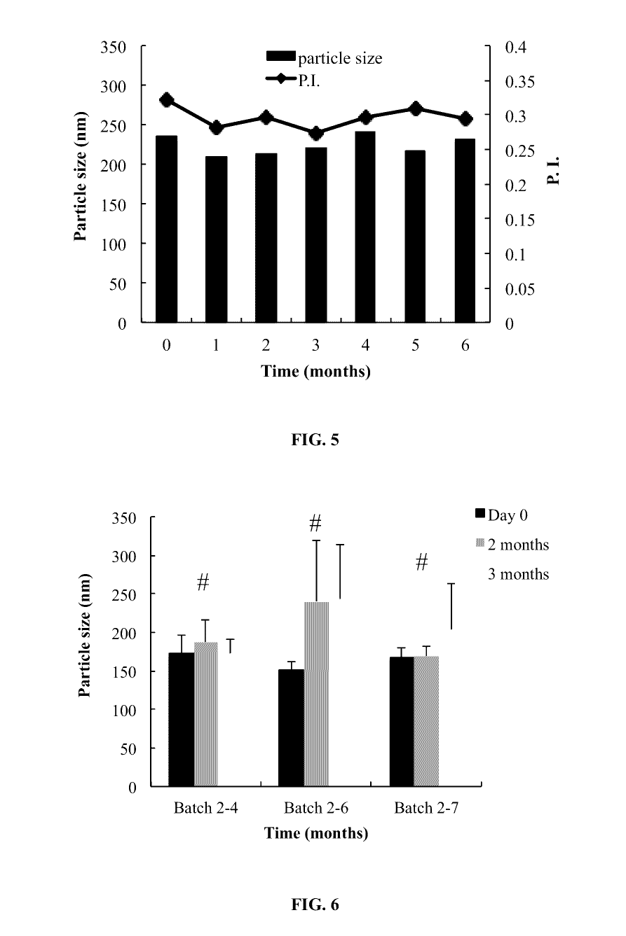

Physical stability studies of NPs. Stability measurement for optimized HDL-mimicking NPs was performed on basis of particle size and. P.I. The batch 4-2 in Table 2D was stable over six months at 4° C. (FIG. 5). Batch 2-4, 2-6 and 2-7 in Table 2B was stable were stable over three months at 4° C. (FIG. 6). The prototype HDL-mimicking α-tocopherol-coated NPs (batch 5-8 in Table 4) were stable over two months at 4° C., and the NGF HDL-mimicking NPs were stable over one month at 4° C. However, considering degradation potentials of both Apo A-I and NGF during long-term storage in aqueous solutions, the inventors are also studying the lyophilization of the NGF NPs to make them as powders for long-term storage. The stability results demonstrated that the NPs developed in this study were stable with or without Apo A-I. The inventors developed not only the NGF HDL-mimicking α-tocopherol-coated NPs but also the stable lipid NPs that did not contain Apo A-I by using Taguchi array. These lipid NPs will be further characterized and evaluated for their potential applications for drug delivery.

The inventors also demonstrated that docetaxel (an anti-cancer drug) could be encapsulated into the HDL-mimicking NPs. This indicates the NPs described herein have potential to deliver not only small molecules but also large molecules. Also, even without Apo A-I the inventors were able to generate stable NPs. Therefore, the novel NPs can be considered as lipid NPs (without Apo A-I), but also as HDL-mimicking NPs that could take advantage of HDL NPs. These unique properties of the NPs developed in this invention will broaden their applications as drug delivery systems to treat various diseases, such as the CNS disorders, cancers and eye diseases.

### Prophetic Example 2

### LR-Targeted NPs to Treat Docetaxel Resistant Metastatic Prostate Cancer

The inventors will evaluate the synergistic efficacy of prostate cancer specific targeted nanoparticles (NPs) containing both docetaxel (DTX) and an antisense oligonucleotide (ASO) to overcome DTX resistance in metastatic castration-resistant prostate cancer (mCRPC).

Novel NPs to Encapsulate Both DTX and OGX-011

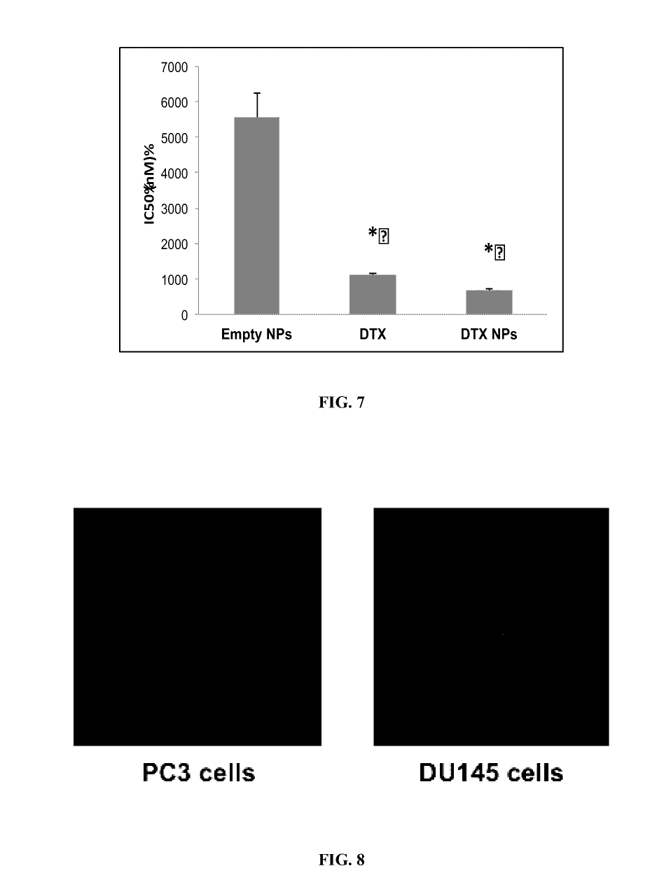

The novel NP delivery system should incorporate DTX and ASO into one NP. As described in Example 1, the inventors have recently developed the novel high-density lipoprotein (HDL)-mimicking NPs to encapsulate nerve growth factor (NGF). The novel NPs have a narrow particle size distribution (polydispersity index, <0.3). Their particle size (<200 nm) is ideal to avoid hepatocytes uptake in liver (particles <100 nm) and splenic filtration (particles 250 nm), but take the advantage of EPR effect (particles 100-200 nm) (Chen and Weiss, 1973 and Huang and Liu, 2011). In this new formulation, NGF formed an ion-pair complex with protamine and then the complex was encapsulated into the lipid core of the HDL NPs. Similar with NGF, OGX-011 will form an ion-pair complex with a positively charged polymer to facilitate the entrapment efficiency of OGX-011 in the NPs. Different from natural HDL, the inventors added TPGS into the NPs to further stabilize HDL-mimicking NPs and improve Apolipoprotein A-I (Apo A-I) entrapment efficiency. Interestingly, the inventors found that the novel NPs were stable at least for 3 months at 4° C. even without Apo A-I. 10% DTX (w/w, drug/total excipients) with >75% entrapment efficiency were successfully loaded into the novel NPs (without Apo A-I), DTX NPs significantly decreased the IC₅₀ of DTX in DTX-resistant prostate cancer cells compared to free DTX (FIG. 8), which proved the uptake of the NPs in cancer cells.

R11 for Targeting Prostate Cancer and Gene Delivery

Additionally, pegylated R11-coated DTX-ASO NPs should provide a prolonged circulation of DTX and ASO compared with free DTX and free ASO. Encapsulation of ASO into the core of the NPs will prevent the degradation of ASO in the blood. Coating PEG on NP surface will create a highly solvated polymer layer at the NP surface, which causes a steric exclusion against the opsonin protein binding and consequently reduces the reticuloendothelial system (RES) uptake. However, the amount of PEG coated on NPs is critical for the steric exclusion (Huang and Liu, 2011). Based on the preliminary data on pegylated PX BTM NPs, 10% of PEG may be appropriate to provide a long circulation of NPs but also release the carried drug efficiently.

Despite widespread reports of in vitro and in vivo results with actively targeting NPs, many studies finished with incomplete characterization of the NPs (Juliano et al., 2014). The amount of targeting ligands, physical and chemical properties, the impact of conjugation on ligand affinity, and pharmacokinetics of the targeted NPs remain largely uninvestigated. The information is important for reproducibility and application of active targeting strategies. Therefore, the inventors will fully characterize Brij 700-R11 conjugate and R11-coated DTX-ASO NPs.

Engineer Pegylated R11-Coated DTX-ASO NPs and Evaluate Them In Vitro

Proposed Methods'and Materials. The inventors will prepare and characterize pegylated R11-coated NPs. Following the promising results from Brij 700-TGF-α conjugation, the inventors will tresylate the —OH group of Brij 78, and then tresylated Brij 700 will react with the N-terminal amine group of R11. Briefly, Brij 700 will be dissolved in dichloromethane and tresyl chloride and pyridine will be added to Brij 700 solution by a drop-wise method at 0° C. The reaction solution will be stirred for 18 hours at room temperature under N₂. Then, the organic solvents will be removed by a rotary evaporator and the precipitates will be purified by acidized ethanol. To prepare Brij 700-R11 conjugate, 100:1 (molar ratio) of tresylated Brij 700 and R11 will be mixed and dissolved into 0.1 M HEPES buffer (pH 7.4). Brij 700-R11 conjugate will be separated and purified using a Sephadex G-25 column. PAGE gel will be used to confirm the purity of the conjugate. The final concentration of R11 in the purified Brij 700-R11 will be measured by Bradford assay.

The inventors will coat both Brij 700-R11 and DSPE-PEG-2000 on the surface of the NPs. Briefly, phosphatidylcholine (PC), sphingomyelin (SM), phosphatidylserine (PS), cholesteryl oleate (CO), and TPGS will be dissolved in ethanol and mixed and dried under N₂. One milliliter of water will be added into the mixture. After 5-min homogenization, the inventors should get the NPs with particle size about 200 nm. And then, a mixture of Brij 700-R11 and DSPE-PEG-2000 will be added into the NPs and incubated at 30° C. for 15 min to coat the conjugate and PEG on the surface of the NPs. The NPs will be put into Amicon Ultra (MW cutoff 100 KDa) to separate free components and encapsulated ones. Free R₁₁ and DSPE-PEG-2000 in the filtrate will be measured using Bradford assay and HPLC with a refractive index detector, respectively, to determine entrapment efficiencies. The NPs will be labeled with BODIPY by incorporating cholesteryl BODIPY 542/563 C11 (Life Technology) into the NPs. Prostate cancer cell lines including DTX-resistant DU145, PC-3 KD1 and C4-2 Neo will be treated with BODIP-loaded R11-coated NPs for 30 min. The bioactivity of the pegylated R11-coated NPs will be determined based on the uptake of the fluorescence (BODIPY) in the cells. Additionally, the location of BODIP will be determined using fluorescence microscopy to evaluate if BODIP presents in the cytosol. The ratio and amounts of Brij 700-R11 and DSPE-PEG-2000 will be optimized to provide the optimal uptake and also about 10% of DSPF-PEG-2000 on the NPs.

Next, the inventors will prepare and characterize pegylated R11-coated DTX-ASO NPs. Different polyanions, such as polylysine and protamine, will be tested to form a suitable ion-pair complex with OGX-011. Briefly, OGX-011 will be mixed with a polyanion at different ratios. Particle size and zeta potential of the complexes will be measured. The optimal ratio will provide particles with zeta potential about 0, indicating the neutralization of the charge on OGX-011. The optimal complex will be added into the R11-coated NPs described above. Briefly, PC, SM, PS, CO and TPGS will be mixed and dried. The complex will be added into the dried mixture and mixed for 20 min. After this, 1 ml water will be added into the mixture and homogenized to form the NPs. And then, Brij 700-R11 will be added into the NPs as described above. To make pegylated NPs, a mixture of Brij 700-R11 and DSPE-PEG 2000 will be added into the NPs.

Particle size, P.I. and zeta potential will be measured by Delsa Nano HC (Beckman Counter). A short-term physical stability will be evaluated based on particle size at 4° C. for 3 months. To measure the entrapment efficiencies of DTX and OGX-011, free DTX and OGX-011 will be separated from the NPs by centrifugation using Amicon Ultra (MW cutoff 100 KDa) at 4° C. The free DTX in the filtrate will be measured by HPLC. Free OGX-011 in the filtrate will be analyzed using a specially validated ELISA/cutting method that was used in Phase I study of OGX-011 (CTBR Bio-Research Inc., Canada) (Chi et al., 2005). The in vitro release of DTX and GU81 from pegylated R11-coated DTX-ASO NPs will be performed using Amicon Ultran (MW cutoff 100 KDa). Briefly, 200 μl of the NPs will be added into 20 ml PBS buffer and shake at 135 rpm over time at 37° C. At certain time intervals, released DTX and OGX-011 will be separated from the NPs using Amicon Ultran and measured as described above. In parallel, the sample will be taken to measure particle size to evaluate physical stability of the NPs in PBS buffer at 37° C. To further mimic the release in the blood circulation, the release study will be also conducted in the whole blood as reported previously (Feng et al., 2013). Briefly. The NPs will be mixed with the fresh mouse blood and incubated for 24 hours at 37° C. with shaking. At a certain time point, 250 μl of blood will be withdrawn to get the plasma. A 15 cm Sepharose CL-4B column (GE Healthcare, US) will be used to separate released Brij 78-R11, DTX and OGX-011. from the NPs. Free Brij 78-R11, DTX and OGX-011 will pass through the column to determine which fractions contain the agents. The corresponding fractions will be collected to measure the released agents. Released Brij 78-R11 will be measured by HPLC as reported previously with modification (Miklan et al., 2009). Released DTX will be measured using PX as an internal standard by an Agilent G6460 Triple Quad LC-MS/MS as described previously [30]. Released OGX-011 will be determined by the ELISA/cutting method as describe above.

The overall criteria for the final pegylated R11-coated DTX-ASO NPs include (1) particle size <200 nm, (2) P.I.<0.3 (monodispersed), (3) entrapment efficiency >80% with minimum drug concentrations of 150 μg/ml for DTX and 100 μg/ml for GU81, (4) physical stability based on particle size for one month at 4° C. and 24 hours at 37° C., and (5) less than 50% release of Brij 78-R11, DTX and OGX-011 within 8 hours in PBS or the blood.

The amounts of DTX and OGX-011 in the NPs will be calculated based on animal studies available in literatures and also the animal studies as described below. It is critical that the targeting ligand can stay on the NPs with the drug for a period of time to achieve tumor accumulation. The in vitro release studies will test this property and assist further NP optimization. All related analytical methods either reported in literature or developed by the inventors will be utilized. If rapid releases are observed, the inventors will change the NP composition using the compositions generated from previous Taguchi array. Also, the inventors may use different polyanions (i.e. hyaluronic acid) to condense OGX-011 and form a stable complex.

The inventors will also evaluate pegylated R11-coated DTX-ASO NPs in prostate cancer cells. They will use DTX-resistant DU145 (androgen receptor [AR] negative) and several DAB2IP-knockdown prostate cancer cell lines generated by Dr. Hsieh. DAB2IP is characterized as a potent tumor suppressor in prostate cancer progression and the loss of DAB2IP is associated with chemoresistance of mCRPC (Wu et al., 2013). These DAB2IP-knockdown (KD) cell lines showed significantly high resistance for DTX, and also upregulated sCLU gene expression (Wu et al., 2013). Among these cell lines, PC-3 (AR negative) and C4-2 (AR positive) have been characterized to express LR and used to test R11 uptake. Specifically, six cell lines will be used for this project: DU145 control cell line and resistant cell line, PC-3 control cell line and resistant cell line (KD1), and C4-2 control cell line (D2) and resistant cell line (Neo). OGX-011 will be fluorescently labeled with Cy3 to assist characterization of OGX-011 in prostate cancer cells. To determine the subcellular localization of OGX-011, cells will be treated with the NPs for 30 min. After fixation, cells will be counterstained with DAPI. The cellular distribution of Cy3-OGX-011 will be examined under fluorescence microscope. Cytotoxicity of the NPs will be measured using MTT assay at 72 hours and compared to controls including the empty pegylated R11-coated ASO NPs, the mixture of the empty NPs with DTX and ASO, pegylated R11-coated DTX NPs and pegylated DTX NPs. Cell apoptosis after treated with pegylated R11-coated DTX-ASO NPs will be tested using in situ Cell Death Detection Kit POD (Roche Applied Science). sCLU expression on the treated cells with OGX-011 and/or DTX will be assessed by Western blotting as reported previously (Sowery et al., 2008).

With well-controlled NP preparation in previous Tasks, the inventors expect that Cy3-OGX-011 will be located in the cytosol. OGX-011 will decrease the gene expression of sCLU in the resistant cells. Pegylated R11-coated DTX-ASO NPs will show superior cytotoxicity compared to DTX NPs and free DTX in the resistant cells. All bioassay methods are available for the studies and the inventors do not expect the problems on them. If OGX-011 does not present in the cytosol, instead of coating PEG and R11 on the NPs, the inventors will coat Apo A-I on the NPs to make HDL-mimicking NPs. HDL provide significant opportunities as gene delivery vehicles because they are endogenous carriers of miRNAs. Data has previously demonstrated that reconstituted HDL NPs escaped endosome and facilitated high efficient systemic delivery of siRNA in vivo (Shahzad et al., 2011 and McMahon et al., 2014), Since HDLs are natural NPs in the body, HDL-mimicking NPs have potential to escape the RES, leading to a long circulation in the blood. Scavenger receptor type B-I (SR-BI) is responsible for natural HDL uptake. Among the normal tissues, only liver has high SR-BI expression, whereas others have minimal to no expression (Shahzad et al., 2011). However, SR-BI overexpresses in cancer cells. Thus, DTX-ASO HDL-mimicking NPs will still have actively targeting effect in cancer cells.

Evaluate Pegylated R11-Coated DTX-ASO NPs In Vivo

For animal studies, all samples will be sterilely prepared in 10% lactose to make them isotonic. A unique feature for the NPs is that concentrated NPs can be made by increasing the amount of each component in the NPs at least 20 times. The maximal tolerate dose of single dose of DTX in mice was reported as 15-33 mg/kg (Dykes et al., 1995). Ten mg/kg of OGX-011 was a safe dose for mice (Sowery et al., 2008). Thus, the inventors will select three doses for subcutaneous (s.c.) models based on in vitro IC50s to fit the range of 3-10 mg/kg of DTX and OGX-011. To meet the dose requirement, concentrated NPs will be prepared to allow 100 μl of i.v. injection in mice.

The inventors will first evaluate pharmacokinetics (PK) of pegylated R11-coated DTX-ASO NPs in mice. PK studies will be performed in BALB/c mice. Five male BALB/c mice (4-6 weeks of age) will receive i.v. injection for each treatment through the tail vein. The inventors will treat mice with 100 μl of pegylated R11-coated DTX-ASO NPs (1 mg/ml of docetaxel and 1 mg/ml of OGX-011) to give a dose of 5 mg/kg for both agents. At given time intervals (0,1, 2, 3, 4, 6, 9 and 24 hours), mice will be sacrificed for blood and tissue collection. The plasma will be divided to two portions to measure DTX and OGX-011 separately. Measurement of DTX in the plasma will be conducted by a LC-MS/MS as reported previously (Kim et al., 2013). OGX-011 will be directly quantified from the plasma using the ELISA/cutting method as mentioned in Task 1.2. The PK parameters will be calculated with standard noncompartmental analyses using Phoenix WinNonlin version 6.3 (Certara, St. Louis, Mo.). The Cₘₐₓ, Iₘₐₓ, T_{1/2}, CL, AUCₗₐₛₜ, and AUC_{0-∞} will be calculated and compared to the controls including DTX, OGX-011, and R11-coated DTX-ASO NPs.

The inventors expect a prolonged circulation of DTX and OGX-011 by using pegylated R11-coated DTX-ASO NPs. The initial loading of PEG will be 10%; however, the inventors will optimize the PEG loading based on the PK results.

Next, the inventors will perform in vivo anti-cancer efficacy, biodistribution and toxicity studies. A s.c. model will be used for dose-finding experiments. An effective dose required to produce synergistic effect of DTX and OGX-011 in vivo will be determined by injecting three doses of pegylated R11-coated DTX-ASO NPs (3, 5, and 10 mg/kg of DTX and OGX-011) in prostate cancer bearing mice (n=8) (See Vertebrate Animals). Mice will be treated once a week for three weeks when the tumor volume reaches 50 mm³. Tumor size and mice will be weighted every three days. At the end of the study, mice will be sacrificed and tumor, kidney, lung, heart, liver and spleen will be flash-frozen in liquid nitrogen. One third of tissues will be fixed for routine histological examination to evaluate the toxicity. One third of tumors will be used to study for tumor immunohistochemical staining as described previously (Sowery et al., 2008). The rest of tumors and tissues will be sonicated in RIPA buffer with a protease inhibitor. The total cell lysate will be use to assess clusterin expression in tumors by Western blotting, OGX-011 concentration in tumors and tissues by the ELISA/cutting method and DTX concentration in tumors and tissues by a LC-MS/MS as described above. Saline, pegylated R11-coated NPs and pegylated DTX-ASO NPs will be used as controls.

To make the proposed studies relevant to the clinic setting, therapeutic efficacy of the NPs will be evaluate in bone metastatic models in SCID mice. Since mCRPC can be both AR positive and AR negative, the inventors will use PC-3 KD1 (or DTX-resistant DU145; AR negative) and C4-2 Neo cells (AR positive) to establish the bone metastatic models. The detailed procedures on animal models are described in the section of Vertebrate Animals. Mice will be injected with the optimal dose selected from Task 2.2 above. Total 7 treatment groups (See Vertebrate Animals) include saline, Texotere, empty NPs, pegylated R11-coated DTX NPs, pegylated DTX-ASO NPs, pegylated R11-coated DTX-ASO NPs, and a mixture of pegylated R11-coated DTX NPs and ASO. To trace tumor growth, the inventors will monitor serum PSA levels (for AR positive tumor) and MRI to observe any delay of relapse. The inventors will harvest tumor biopsies starting the end of last treatment and every two weeks for histologic examination. The inventors will also determine the activity of bone stromal cell using Von Kossa staining, immunostaining for osteopontin or X-ray for osteoblastosis. In this study, the inventors will also document the PSA-free survival based on the recurrent time of PSA and the survival rate (Kaplan-Meier curve) based on the time of animal sacrifice (i.e., BLI intensity)

The inventors will decide which cell, PC-3 KD or DTX-resistant DU145, will be used for the metastatic model based on the outcome of other studies. Enhanced synergistic efficacy is expected from pegylated R11-coated DTX-ASO NPs compared to the controls, especially the mixture of pegylated R11-coated DTX NPs and OGX-011. sCLU expression in the group of pegylated R11-coated DTX-ASO NPs will be lower compared to the controls. Also, the inventors expect the actively targeting outcome—enhanced accumulation in tumor by R11-coated NPs compared with uncoated NPs. If the proposed bioassay methods are not sensitive enough, the inventors will use radio-labeled DTX and OGX-011 for the biodistribution study. Except of Brij 700, the components in the NPs are FDA-approved excipients and naturally exit in human body; thus, toxicity is not expected from the empty NPs. Since OGX-011 will be co-delivered with DTX by i.v. injection in the NPs, the inventors expect that a low dose of OGX-011 may generate the synergistic effect. All tumor models in this example have established in prior studies. The s.c. model is used not only for dose finding but also for NP development. If s.c. model does not yield results, the inventors will further optimize the NPs using the strategies as described above and repeat the animal studies.

### Example 3

### Additional Applications of Nanoparticles

Small Molecules

Docetaxel (DTX) was dissolved in ethanol at 200 kg/ml. Phosphatidylcholine (PC), sphingomyelin (SM), phosphatidylserine (PS), cholesteryl oleate (CO) and D-α-Tocopheryl polyethylene glycol succinate (TPGS) were dissolved in ethanol to prepare stock solutions at 1 mg/ml, respectively. The, 59 μl PC, 11 μl SM, 4 μl PS, 15 μl CO, 45 μl TPGS and 75 μl DTX were added into a glass vial. After mixing, the ethanol is removed under a gentle nitrogen stream. The mixture was homogenized at 8600 rpm for 5 min at room temperature to form DTX NPs. DTX NPs were characterized by measuring particle size, size distribution, entrapment efficiency.

DTX-resistant castration-resistant prostate cancer cells (DU145 cells) were treated with different concentrations of free DTX and DTX NPs, respectively. After 72 hours, MTT assay was used to measure cell viability and the IC₅₀s of free DTX and DTX NPs were calculated.

The inventors successfully loaded 10% docetaxel (DTX) (w/w, drug/total excipients) with >75% entrapment efficiency into the novel NPs (without Apo A-I). Particle size (˜170 nm) of DTX NPs and size distribution were similar with the original NPs. According to the cytotoxicity studies, DTX NPs significantly decreased the IC₅₀ of DIN in DTX-resistant CRPC cells compared to free DTX (FIG. 7), which also proved the uptake of the NPs in cancer cells.

Proteins

Materials and Cell Culture. Protamine from salmon, protamine grade X, protamine sodium salt USP, poly-lysine and cholesteryl oleate (CO), Sodium chloride, sodium acetate, Triton X-100, bovine serum albumin (BSA), phosphate buffer saline (PBS), phenylmethylsulfonyl fluoride (PMSF) and benzethonium chloride were purchased from Sigma (St. Louis, Mo.), Sephadex G-50, Sephadex G-100, Sephacryl S-100 and Sepharose CL-4B were also purchased from Sigma-Aldrich (St. Louis, Mo.). PC, SM, and phosphatidylserine (PS) were purchased from Avanti polar lipids (Alabaster, Ala.). TPGS was provided by BASF as a gift. Apo A-I was purchased from Athens research and technology (Athens, Ga.). Recombinant human NGF was purchased from Creative Biomart (Shirley, N.Y.). Bradford reagent was obtained from Thermo Scientific (Rockford, Ill.). Amicon ultra centrifugal filters (0.5 mL) were obtained from Merck Millipore (Germany). Float-A-Lyzer G2 Dialysis device (MWCO 300 kDa) was purchased from Spectrum Laboratories (Rancho Dominguez, Calif.). Human beta-NGF DuoSet ELISA kit was purchased from R&D Systems (Minneapolis, Minn.).

Animals. Bcl mice (adult males, 25˜30 g) were purchased from Charles River Laboratories (Wilmington, Mass.). All animal experiments were carried out under an approved protocol by the Institutional Animal Care and Use Committee at the University of North Texas Health Science Center.

Optimization of preparation procedure for prototype HDL-mimicking NPs. Blank HDL-mimicking NPs were prepared by a self-assembly method. To maintain NGF bioactivity after NP preparation, we chose low temperature (50° C.) or room temperature for preparation. All excipients were dissolved in ethanol to prepare stock solutions. PC (43.1%), SM (8.1%), PS (2.7%), CO (7.7%) and TPGS (38.4%) (percentages based on w/w) were added into a glass vial to form a thin film after removing ethanol by nitrogen. And then 1 ml of milliq water was added into the vial. Five different procedures were evaluated to hydrate the film to form NPs, including: 1) adding water at 50° C. and stirring at 50° C. for 30 min at 600 rpm, 2) adding water at 50° C.; and stirring at room temperature (RT) for 30 min at 600 rpm, 3) adding water at RT and stirring at RT for 30 min at 600 rpm, and 4) adding water at 50° C. and homogenizing 5 min using a homogenizer at 8600 rpm, and 5) adding water at RT and homogenizing 5 min using a homogenizer at 8600 rpm. To further evaluate the influence of homogenization time on NP formation, the mixtures were homogenized for 0, 1, 2, 3, 4, 5, and 6 min after adding water at RT. After preparation, particle size and polydispersity index (P.I.) of NPs were measured using a Delsa Nano HC particle analyzer (Beckman Coulter, Calif.) at 90° light scattering at 25° C.

Development of prototype HDL-mimicking NPs. Nanoparticles without Apo A-I. PC, SM and PS were selected as phospholipid components and CO was selected as the lipid component to develop the HDL-mimicking NPs. To simplify the design and quickly find the optimal compositions, we considered phospholipids as one variable that include PC, SM and PS. The percentage of each phospholipid in the total phospholipids excluding CO was fixed as PC (76%), SM (14%) and PS (10%), which is close to the composition of phospholipids, but doubled the amount of PS, compared to the composition of natural HDLs. To evaluate different ratios of phospholipids and CO, the inventors designed two arrays. In the array #1 (Table 2A and 2B, above), the ratios of total phospholipids and CO were controlled in a range of 0.6 to 1.6 (total phospholipids/CO, w/w). This array for 3 levels 2 variables (phospholipids and CO) was used to give three different concentrations for each excipient. The array #2 (fable 2C and 2D, above), an array for 2 levels 2 variables, was used to give the different ratios of total phospholipids and CO in the range of 4.9 to 14 (total phospholipids/CO, w/w). In the array #2, the percentage of each phospholipid in the total phospholipids excluding CO was fixed as PC (80%), SM (15%) and PS (5%). NPs were prepared as described above. After forming the thin film, 1 ml of milliq water at RT was added into the vial and homogenized for 5 min at 8600 rpm to form NPs. To make TPGS-coated NPs, certain amounts of TPGS were added into the compositions in Tables 2B and 2D to give a total surfactant (phospholipids+TPGS) in a range of 60 μg/ml to 120 μg/ml. Particle size and P.I. were measured as described above.

Optimization of loading Apo A-I in the prototype HDL-mimicking NPs. Based on the particle size and size distribution, the optimal compositions were selected to load Apo A-I, which are highlighted in Tables 2A-D. After homogenization for 5 min as described above, a certain amount of Apo A-I was added into each composition (Table 3, above). Four different conditions, including 2-hour stirring at RT, 4-hour stirring at RT, 4-hour stirring at RT followed with incubation at 4° C. overnight, and 4-hour stirring at RT followed with stirring at 4° C. overnight, were evaluated to load Apo A-I. Particle size and size distribution were measured as described above. EE of Apo A-I was analyzed by ultrafiltration. Briefly, 0.2 ml of the NPs were added into Amicon Ultra (Molecular cutoff 100 KDa) and centrifuged at 14000 rpm at 4° C. for 3 min. After this, 400 μl water were added into the insert of Amicon to wash the membrane with the same centrifugation condition. Apo A-I was passed through the membrane and washed with the same approach as described above to measure the recovery of Apo A-I in this separation method. The concentration of unloaded (free) Apo A-I in the filtrate was measured by Bradford assay, Loading and EE of Apo A-I were calculated as follows:

% loading=(drug added into NP)/(total weight of excipients+drug)×100% Eq. (1)

% EE=(1−unloaded drug/total drug added into NP)×100% Eq. (2)

Further optimization on Apo A-I loading was studied based on the composition of the batch 4-2. To optimize Apo A-I loading, different amounts of Apo A-I were added into the NPs (Table 4, above) by changing the amount of PC, but keeping the same amounts of SM, PS, CO and TPGS in the batch 4-2. Loading and EE of Apo A-I were measured and calculated as described above.

Particle size stability of prototype HDL-mimicking NPs at 4° C. The physical stability of the prototype HDL-mimicking NPs was assessed over time at 4° C. Prior to particle size measurement, NPs were allowed to equilibrate to RT. One milliliter of NPs was used to measure the particle size and P.I as described above.

Development of NGF-loaded HDL-mimicking NPs/Optimization of ion-pair complex for NGF. To efficiently load NGF into the NPs, poly-lysine and three types of protamines were tested to form an ion-pair complex with NGF. Protamines included protamine from salmon, protamine grade X and protamine sodium salt USP. Poly-lysine, protamines and NGF were dissolved in water at the concentration of 1 mg/ml. NGF was added into poly-lysine or protamine solutions at 0.8:1, 1:1, and 1:1.2 ratios (NGF:polymer, w/w). The complex was allowed to stand at RT for 10 min, and then diluted with 1 ml of water or PBS to measure particle size as described above and also to measure zeta potential using the particle analyzer. The optimal ratio of the complex was determined according to particle size and zeta potential.

Preparation of NGF-loaded HDL-mimicking NPs. Poly-lysine and protamine USP were selected to prepare NGF-loaded NPs. Briefly, 10 μg of NGF was mixed with 10 μg poly-lysine or protamine USP (1:1, NGF:polymer, w/w) and kept for 10 min at RT to form the complex. PC, SM, PS, CO and TPGS ethanol solutions (Table 6; above) were mixed and then ethanol was removed by nitrogen to form the thin film as described above. Two procedures were tested to add the NGF complex into NPs. In the first procedure, the NGF complex was added into the thin film, and then 1 ml of water at RT was added and homogenized for 5 min to incorporate NGF. In the second procedure, 1 ml of water at RT was first added into the thin film and homogenized for 5 min; and then the NGF complex was added into the solution. After the addition of NGF complex, the solution was incubated at 37° C. for 30 min, and then stirred at RT for 30 min until cooling in order to incorporate NGF. The defined amount of Apo A-I was added into each solution and stirred at RT overnight to form the final NGF-loaded HDL-mimicking α-tocopherol-coated NPs. Particle size and zeta potential were measured as described above.

Determination of NGF entrapment efficiency in NGF-loaded IDOL-mimicking NPs. Gel filtration chromatography was used to separate unloaded NGF from NGF NPs. To determine the fractions containing NGF, 200 μl of NGF solution (10 μg/ml) were added on a Sepharose 4B-CL column and eluted with PBS. Twelve fractions (about 1 ml for each) were collected and measured for the concentrations of NGF using a Sandwich ELISA method developed based on a Sandwich ELISA kit for NGF. In a separate experiment, 200 μl of NGF HDL-mimicking NPs were eluted from the same column. The intensity in each fraction was measured using the particle analyzer to determine fractions containing NPs. The concentrations of NGF in fraction 5 to fraction 10 were measured and added together to calculate the amount of unloaded NGF. Loading and EE of NGF were calculated using equation (1) and (2) as described above.

In vitro release study. The release of NGF from NGF NPs (n=4) was studied using a dialysis method. The release medium was PBS (pH 7) containing 5% BSA to mimic the physiological condition in blood. Briefly, 200 μl NGF NPs and 400 l release medium were loaded into the dialysis tube (invco 300 kDa). Then the dialysis tube was placed into a 30 ml release medium and shaken at a 37° C. at 135 rpm. At the time intervals (1, 2, 4, 6, 8, 24, 48 and 72 hours), 100 μl of the release medium were withdrawn and replaced with an equal volume of fresh medium. The amounts of released NGF in the medium were analyzed by a NGF Sandwich ELISA kit. As a control, free NGF (n=4) was studied in parallel.

Tissue distribution of NGF NPs. Mice were randomly divided to three groups (n=3). Saline, free NGF and NGF NPs were injected, respectively, through tail vein at a dose of 40 μg/kg for each group. After injection, mice were sacrificed at 30 min, and blood, brain, liver, spleen and kidney were collected. Blood samples were centrifuged at 3400 rpm at 4° C. for 5 min to obtain plasma. Plasma and tissues were stored at −80° C. until analyzed. For tissue samples, 100 mg of tissues were suspended in a 10-times volume of extraction buffer (0.05M sodium acetate, 1.0 M sodium chloride, 1% Triton X-100, 1% BSA, 0.2 mM PMSF, and 0.2 mM benzethonium chloride) and homogenized at 4° C. The concentrations of NGF in plasma and tissues were measured by the Sandwich ELISA kit.

Statistical Analysis. Statistical analysis of the data including ANOVA and t-test, wherever needed, was performed using Graph Pad Prism software. Results were considered significant if p<0.05.

Results—in vitro release study. The release profiles of free NGF and NGF NPs are shown in FIG. 14. Free NGF passed through the membrane readily and reached 83% in the first hour. The inventors observed the tendency of NGF to bind with the membrane when we tested the entrapment efficiency. In the release studies, they added 5% BSA to reduce the binding of NGF as well as matching the BSA concentration in blood. The result indicated that 5% BSA efficiently prevented the binding of NGF to the membrane. With this advance, the inventors can accurately measure the released NGF from NGF NPs. NGF NPs showed a slow release without a burst release. Only 5.5% of NGF was released within 1 hour. The release of NGF reached a plateau at 8 hours (9.9%) and kept over 72 hours. The release results demonstrated that NGF was entrapped in the core of the NPs, which aligns with the result of the entrapment efficiency.

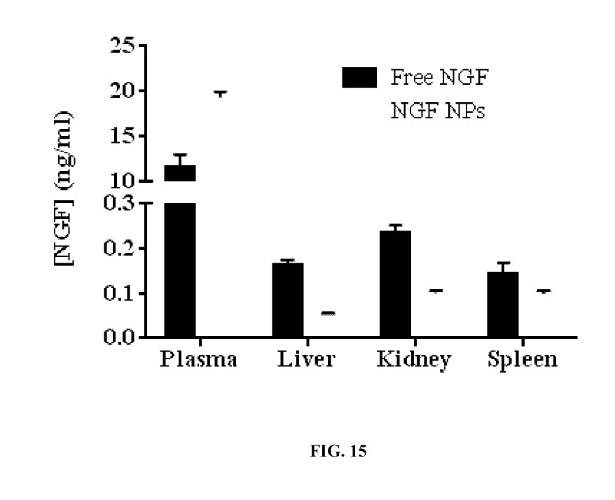

Biodistribution. One of the inventors' hypotheses was that NPs can protect NGF from degradation and control NGF release in order to improve the half-life of NGF after intravenous injection. Hence, they measured the biodistribution of NGF NPs in mice. As shown in FIG. 8, NGF NPs increased the plasma concentration of NGF by 1.7-fold compared to free NGF. For tissues, NGF NPs decreased the tissue uptake by 3-fold in liver, 2.3-fold in kidney and 1.4-fold in spleen. The results demonstrated that the NPs prolonged the circulation of NGF in blood. As shown in the release studies (FIG. 14), NGF was entrapped inside the NPs and slowly released from the NPs. Thus, the NPs protected NGF from degradation in vivo, leading to a long circulation in blood and reduced uptake in tissues (FIG. 15). When the NPs are used to deliver NGF to brain, the prolonged circulation would provide more opportunity for the brain uptake compared to free NGF. Therefore, the novel HDL-mimicking NPs are very promising for delivery of NGF through intravenous injection.

Neurite Outgrowth Study. It is important to maintain protein's activity after the formulation of the NPs. Thus, the inventors chose to measure the bioactivity of NGF HDL-mimicking NPs in PC12 cells for neurite outgrowth. They pre-coated a 6-well plate with rat tail collagen type I. They seeded PC12 cells at a density of 10000 cells/well to the pre-coated 6-well plate overnight to allow cells to attach on the plates. They diluted free NGF (10 μg/ml) and NGF HDL-mimicking NPs (10 μg/ml) with the culture medium to prepare various concentrations at 0.5, 1, 5, 10, 50, and 100 ng/ml using half-half dilution. Then, they added 100 μl of sample into each well of the plate and culture for 4 days. At day 4 they changed the medium to fresh medium containing the corresponding treatment and then continue the treatment for another 3 days. At day 7, they visualized cells by an inverted light microscope and take the imaging from each well at random spots under 10× magnification.

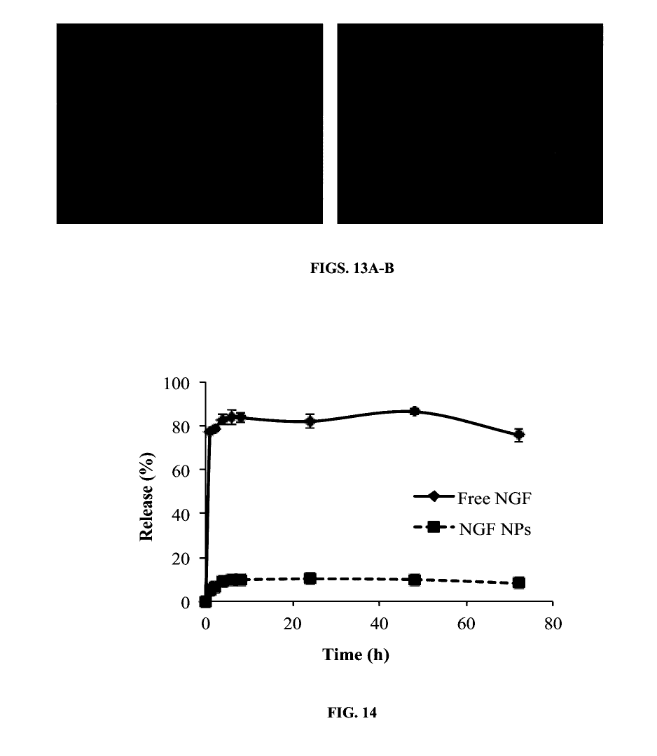

FIGS. 13A-B represent the imaging of neurite outgrowth when the cells were treated with 50 ng/ml of free NGF (FIG. 13A) and NGF HDL-mimicking NPs (FIG. 13B). When the treatment concentration was higher than 10 ng/ml, neurite outgrowth was clearly observed by the microscope. At these high concentrations, free NGF and NGF HDL-mimicking NPs did not show significant difference on the effect of neurite outgrowth. When the concentration of NGF was lower than 10 ng/ml, neurite outgrowth cannot be observed clearly for both free NGF and NGF HDL-mimicking NPs. Thus, the inventors have demonstrated the comparable bioactivity of NGF HDL-mimicking NPs with free NGF.

Micro RNA (Without Apo A-I)

The inventors utilized the novel NPs (without adding Apo A-I) to encapsulate microRNA-363 for prostate cancer. The preparation procedure was similar with that of NGF HDL-mimicking NPs. Briefly, phosphatidylcholine (PC), sphingomyelin (SM), phosphatidylserine (PS), cholesteryl oleate (CO) and D-α-Tocopheryl polyethylene glycol succinate (TPGS) were dissolved in ethanol to prepare stock solutions at 1 mg/ml, respectively. The, 59 μl PC, 11 μl SM, 4 μl PS, 15 μl CO, and 45 μl TPGS were added into a glass vial. After mixing, the ethanol is removed under a gentle nitrogen stream. The mixture was homogenized at 8600 rpm for 5 min at room temperature to form the prototype NPs. The inventors mixed microRNA-363 with protamine (1:2 ratio, w/w) to form the ion-pair complex. Then, they added the complex into the prototype NPs and incubate them at 37° C. for 30 min. After cooling, they obtained microRNA-363 loaded NPs.

The inventors used Cy5 labeled microRNA-363 to prepare the NPs and studied the cellular uptake of the NPs by a confocal microscopy. Cells were seeded in 12-well tissue culture plates at a density of 2×10⁴ cells and incubated overnight at 37° C. Then the cells were treated with free microRNA-363 or microRNA-363 NP at 6.4 μg/ml for 3 hrs at 37° C. The cells were washed with PBS, and then fixed with 4% formaldehyde. The nuclei were stained with DAPI and the cells were mounted to glass slide. The red fluorescence of Cy5 was visualized with a confocal microscope. Cy5-labeled miRNA-363 was successfully encapsulated into the NPs. The particle size of microRNA-363 NPs was ˜170 nm with a narrow size distribution.

Moreover, the uptake study using confocal microscopy showed that miRNA-363 was located in the cytoplasm of PC3 and DU145 cells (FIG. 8). This result demonstrated that the inventors' novel NPs are promising to escape endosome and deliver miRNAs to cytoplasm. In addition, they can lyophilize the NPs without the loss of NP properties, which warrants long-term stability of macromolecules and clinic translation. Therefore, the novel NPs have the ability to incorporate small molecules and macromolecules. The inventors will use their novel NPs to deliver the combination of small molecules and macromolecules, e.g., the combination of microRNA-363 (or microRNA-145) and DTX.

Novel HDL-Mimicking TPGS-Coated NPs Delivering NGF, DTX and miRNA

To encapsulate NGF, the inventors used protamine or poly-D-lysine to form an ion-pair complex with NGF by charge-charge interaction, which normalized the surface charge of NGF to facilitate encapsulation of NGF. The characterization of novel NGF NPs is summarized in Table 7 (above). Note: phosphatidylcholine (PC), sphingomyelin (SM), phosphatidylserine (PS), cholesteryl oleate (CO), vitamin E TPGS (TPGS).

In addition to appropriate particle size and entrapment efficiency, the zeta potential of NGF NPs is negative. Liposomes have been commonly used for gene delivery; however, safe and efficacious delivery in vivo is rarely achieved due to toxicity, nonspecific uptake, and unwanted immune response. The nonspecific response and toxicity are directly linked to the positive charge on the surface of the liposomes necessary for the binding of gene therapeutic agents. Thus, because of negative surface charge, the inventors' NPs will overcome the problems of liposomes. Importantly, novel NGF NPs had the same bioactivity compared to free NGF, demonstrating that encapsulating of NGF into the NPs did not affect the efficacy of NGF.

The inventors also successfully loaded 10% DTX (w/w, drug/total excipients) with >75% entrapment efficiency into the novel NPs (without Apo A-I). DTX NPs significantly decreased the IC₅₀ of DTX in DTX-resistant CRPC cells compared to free DTX (FIG. 7), which proved the uptake of the NPs in cancer cells.

Novel HDL-mimicking TPGS-coated NPs delivering miRNA: Natural HDLs are endogenous carriers of miRNAs. Data demonstrated that reconstituted HDL NPs escaped endosome and facilitated high efficient systemic delivery of siRNA in vivo. The inventors developed their HDL-mimicking NPs based on the composition of natural HDLs (Table 2B, above); and tested the feasibility of their novel NPs to deliver miRNA-363. Similar with NGF NPs, the inventors used protamine to form the complex with miRNA-363 by charge-charge action. Cy5-labeled miRNA-363 was successfully encapsulated into the NPs. Moreover, the uptake study using confocal microscopy showed that miRNA-363 was located in the cytoplasm of PC3 and DU145 cells (FIG. 8). This result demonstrated that these novel NPs are promising to escape endosome and deliver miRNAs to cytoplasm. In addition, the inventors can lyophilize the NPs without the loss of NP properties, which warrants long-term stability of macromolecules and clinic translation. Therefore, the novel NPs have the ability to incorporate small molecules and macromolecules. These novel NPs can be used to deliver miRNA-145 as well as the combination of miRNA-145 and DTX.

SiRNA

Non-viral gene delivery systems, including lipid-based nanoparticles (NPs), polyethylenimine-based delivery system, dendrimers, poly(lactide-co-glycolide) NPs, have been extensively studied. The inventors lipid-based NPs are novel in structure; they mostly like a combination of lipoplexes and HDL NPs. All components in these novel NPs naturally exist and have no toxicity. Instead of using cationic lipids that caused the toxicity of lipoplexes, the inventors used protamine, a FDA-approved excipient, to form an ion-pair complex with macromolecules. By adding TPGS, the inventors were able to simply prepare the NPs by a self-assembly method, addressing the manufacturing difficulty and high cost of the NPs.

The preparation procedure was similar with that of microRNA-363 loaded NPs as described above. Briefly, phosphatidylcholine (PC), sphingomyelin (SM), phosphatidylserine (PS), cholesteryl oleate (CO) and D-α-Tocopheryl polyethylene glycol succinate (TPGS) were dissolved in ethanol to prepare stock solutions at 1 mg/ml, respectively. The, 59 μl PC, 11 μl SM, 4 μl PS, 15 μl CO, and 45 μl TPGS were added into a glass vial. After mixing, the ethanol is removed under a gentle nitrogen stream. The mixture was homogenized at 8600 rpm for 5 min at room temperature to form the prototype NPs. The inventors mixed siRNA with protamine (1:1 ratio, w/w) to form the ion-pair complex. Then, the inventors added the complex into the prototype NPs and incubate them at 37° C. for 30 min. After cooling; they obtained siRNA loaded NPs.

To study the cellular uptake, the inventors used FITC-labeled model siRNA and Cy3-labeled anti-GAPDH siRNA to make the NPs and test them by a confocal microscope. Cells were seeded in 12-well tissue culture plates at a density of 2×10⁴ cells and incubated overnight at 37° C. Then the cells were treated with free microRNA-363 or microRNA-363 NP at 6.4 μg/ml for 3 hours at 37° C. The cells were washed with PBS, and then fixed with 4% formaldehyde. The nuclei were stained with DAPI and the cells were mounted to glass slide. The green fluorescence of FTIC or the yellow fluorescence of Cy3 was visualized with a confocal microscope.

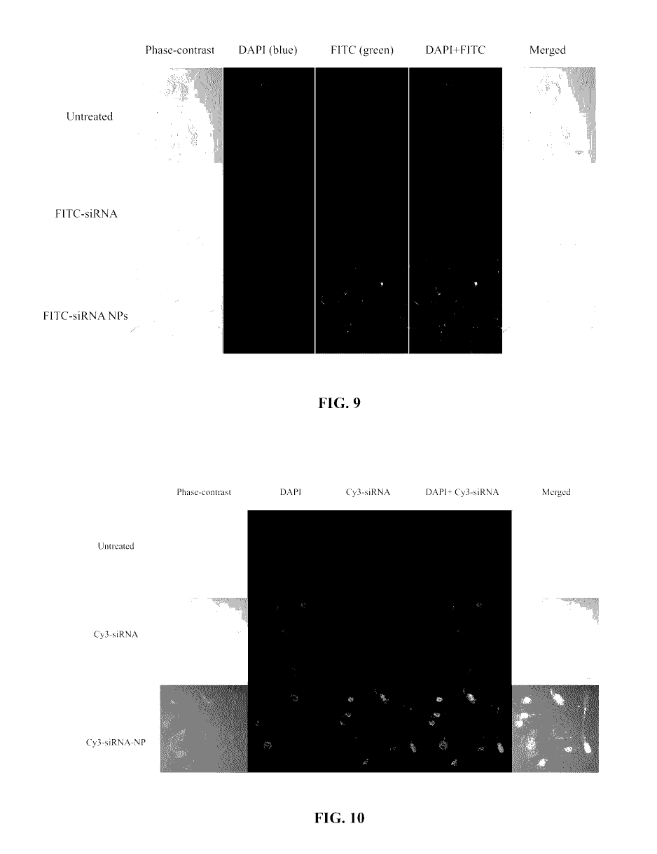

The inventors have encapsulated nerve growth factor (NGF) into the novel NPs. Over 65% of NGF was entrapped into the NPs with 170 nm of particle size. Here, the inventors explored the novel NPs for encapsulation of siRNA. Both fluorescent-labeled siRNAs were successfully encapsulated into the NPs with over 75% entrapment efficiency. The particle size of siRNA NPs was ˜170 nm with a narrow size distribution. Cells treated with siRNA NPs showed internalization and accumulation of green (FTIC, FIG. 9) or yellow (Cy3, FIG. 10) fluorescence in cytosol. In contrast, no fluorescence was observed in cytosol of cells treated with free model siRNA and free anti-GAPDH siRNA. These results demonstrate that the novel NPs are promising to escape endosome and deliver siRNA to cytoplasm for efficient gene transfection,

Use of Endosomal Escaping Agents to Further Modify of NP Composition

Endosomal escaping agents, also call fusogens, including MGDG (monogalactosyldiacylglycerol), diacylglycerol, polyphosphoinositides and fatty acids (e.g., oleic acid and arachidonic acid), may be incorporated into the nanoparticle to enhance gene knockdown.

MGDG is a nonionic lipid and is a non-bilayer lipid; however, it plays a crucial role in membrane fusion. MGDG with conical morphology induces negative curvature, consequently forming inverted hexagonal phase (HII). Thus, MGDG has potential to break endosome membrane to assist genes escaping endosome, and thus incorporated MGDG in the inventors NP composition improve the efficiency of gene knockdown.

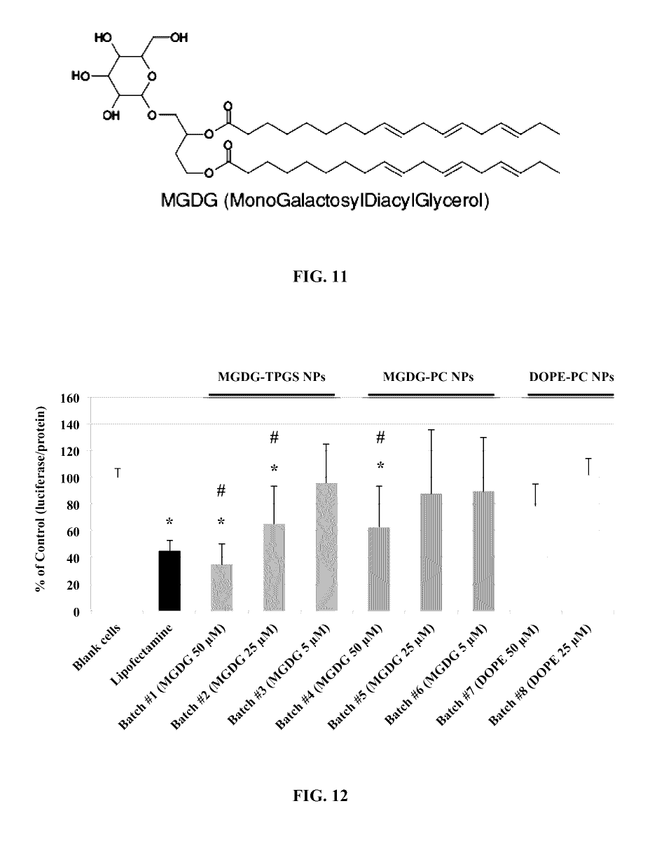

In addition, MGDG has a moiety of sugar (FIG. 11). Instead of using Apo A-I, the inventors included MGDG in the NP composition to prepare MGDG-coated NPs which could act as a “sugar” bead to target to GLUT1 (a glucose transporter in the blood-brain barrier) in order to facilitate across the BBB.

MGDG, TPGS, DOPE and PC were dissolved in ethanol at 1 mg/ml, respectively. The excipients were mixed with certain amounts (Table 8). Ethanol was removed by nitrogen gas. The mixture was homogenized by using a homogenizer at 8600 rpm for 5 min at room temperature to form the prototype NPs. Alternatively, the mixture was sonicated for 1-5 min at room temperature using a sonication probe to form the prototype NPs. Then NGF or siRNA was formed the complex with protamine as described above. The complex was added into the prototype NPs and incubated for 30 min at 37° C. The NPs were characterized for particle size, size distribution and entrapment efficiency.

To test the efficiency of gene knockdown, PC3-Luc+ cells, in which PC3 cells (prostate cancer cells) were stably transfected with luciferase, were seeded in a 96-well tissue culture plate at a density of 8000 cells/well and incubated overnight at 37° C. Nanoparticle was prepared based on the batch compositions listed in Table 9 below. The procedure of preparation is described above. 20 μl of NPs were added to each well with 100 ul culture medium. The final siRNA concentration in each well was 12.3 pmole. After 48 h of the treatment, the medium was removed. Luciferase expression was measured by a luciferase assay. Proteins in each well were measured by a BCA assay. Then, luciferase expression in each well was normalized with protein concentration. The gene knockdown efficiency was represented by the percentage of luciferase/protein comparing with the control (blank cells): % gene knockdown=Treatment (luciferase/protein)/Control (luciferase/protein)×100%

To evaluate the novel MGDG NPs to encapsulate NGF, the inventors prepared NGF MGDG NPs. The compositions of novel NGF NPs and their characterization are shown in Table 8. The NPs had a narrow size distribution. For all batches in Table 8, the entrapment efficiency of NGF or siRNA was over 95%.

To test the efficiency of gene knockdown, the inventors prepared different MGDG NPs to encapsulate anti-luciferase siRNA (Table 9). The results of gene knockdown in PC3-KD1 Luc⁺ cells are shown in FIG. 12. The NPs composed of MGDG and TPGS shows a dose-dependent gene knockdown while changing the concentrations of MGDG. At the MGDG concentrations of 25 μM (batch #2) and 50 μM (batch #1 and batch #4), the NPs significantly decreased the expression of luciferase. Importantly, batch #1, batch #2 and batch #4 did not show significant difference compared to the commercial gene transfection agent (lipofectamine) (#p>0.05), suggesting the great efficiency of the NPs for gene knockdown. Very likely, MGDG induced membrane fusion to facilitate siRNA escaping endosome. According to the results, MGDG has better ability for gene silencing than DOPE. Therefore, the novel NPs in this invention have great potential for gene therapy.

TABLE 8
The compositions and characterization
of the modified NGF NPs containing MGDG

| | PC | TPGS | MGDG | NGF | Protamine | Particle | |
|---|---|---|---|---|---|---|---|
| Batch | (μg) | (μg) | (μg) | (μg) | (μg) | size | P.I. |
| 1 | 20 | — | 60 | 10 | 10 | 149.3 | 0.16 |
| 2 | — | — | 60 | 10 | 10 | 252.3 | 0.063 |
| 3 | 10 | — | 60 | 10 | 10 | 352.3 | 0.211 |
| 4 | — | 10 | 60 | 10 | 10 | 286.7 | 0.141 |
| 5 | — | 20 | 60 | 10 | 10 | 132.6 | 0.231 |

TABLE 9
The compositions and characterization of
the modified siRNA NPs containing MGDG

| | MGDG | TPGS | PC | DOPE | siRNA | Protamine |
|---|---|---|---|---|---|---|
| Batch | (μg) | (μg) | (μg) | (μg) | (μg) | (μg) |
| #1 | 240 | 80 | — | — | 8 | 8 |
| #2 | 120 | 200 | — | — | 8 | 8 |
| #3 | 25 | 295 | — | — | 8 | 8 |
| #4 | 240 | — | 80 | — | 8 | 8 |
| #5 | 120 | — | 200 | — | 8 | 8 |
| #6 | 25 | — | 295 | — | 8 | 8 |
| #7 | — | 80 | — | 240 | 8 | 8 |
| #8 | — | 200 | — | 120 | 8 | 8 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,091,513
U.S. Patent Application Publication No. 2005/0214860
Aloe et al., J. Transl. Med., 10:239-5876-10-239, 2012.
Apfel, Int. Rev. Neurobiol., 50:393-413, 2002.
Balazs et al., J. Neurochem., 89:939-950, 2004.
Barnett, et al., Exp. Neurol., 110:11-24, 1990
Bruce, et al., Neurobiol. Aging, 10:89-94, 1989
Chen and Weiss, Blood., 41:529-537, 1973.
Chi et al., J. Natl. Cancer Inst., 97:1287-1296, 2005.
Dong et al., Eur. J. Pharm. Biopharm., 72:9-17, 2009.
Dykes et al., Invest. New Drugs., 13:1-11, 1995.
Eriksdotter Jonhagen et al., Dement. Geriatr. Cogn. Disord., 9:246-257, 1998.
Feng et al., Adv. Healthc. Mater., 2:1451-1457, 2013.
Goti et al., J. Neurochem., 76:498-508, 2001.
Granholm et al., J. Pharmacol. Exp. Ther., 268:448-459, 1994.
Hu et al., Cancer Res., 56, 3055-3061, 1996
Huang and Liu, Annu. Rev. Biomed. Eng., 13:507-530, 2011.
Iwai, et al., Chem. Pharm. Bull., 34:4724-4730, 1986
Iwane, et al., Biochem. Biophys. Res. Comm., 171:116-122, 1990
Juliano et al., Nucleic Acid Ther., 24:101-113, 2014.
Kanaya, et al., Gene, 83:65-74, 1989
Kim et al., Biomed. Chromatogr., 27:306-310, 2013.
Kurakhmaeva et al., J. Drug Target., 17:564-574, 2009.
Liu et al. Cell Mol. Biol., 49(2):209-216, 2003
Mandel, Curr. Opin. Mol. Ther., 12:240-247, 2010.
McArthur et al., Neurology, 54:1080-1088, 2000.
McMahon et al., Expert Opin. Drug Deliv., 11:231-247, 2014.
Miklan et at., Biopolymers., 92:489-501, 2009.
Olivier, NeuroRx, 2:108-119, 2005.
Pinto Reis et al., Nanomedicine 2:53-65, 2006.
Provost et al., CR Biol. 326:841-51, 2003.
Shahzad et al., Neoplasia., 13:309-319, 2011.
Sofroniew et al., Annu. Rev. Neurosci., 24:1217-1281, 2001.
Sowery et al., BJU Int., 102:389-397, 2008.
Tria et al., Exp. Neurol., 127:178-183, 1994.
Vaishya et al., Expert Opin. Drug Deliv., 12:415-440, 2015.
Wahlberg et al., J. Neurosurg., 117:340-347, 2012.
Wu et al., Clin. Cancer Res., 19:4740-4749, 2013.
Xie et al., J. Control. Release, 105:106-119, 2005.
Zhang et al., Biomaterials 33:4889-4906, 2012.
Zhang et al., Pharm. Res., 26:1561-1580, 2009.

1. A composition comprising:
(a) a therapeutic agent;
(b) an α-tocopheryl compound;
(c) a phospholipid composition; and
(d) a steroid or steroid derivative,
wherein the composition is formulated as a nanoparticle and the α-tocopheryl compound is substantially located on the surface of the nanoparticle.

2. A composition comprising:
(a) a therapeutic agent;
(b) an α-tocopheryl compound;
(c) a phospholipid composition;
(d) a steroid or steroid derivative, and
(e) an apolipoprotein;
wherein the composition is formulated as a nanoparticle and the α-tocopheryl compound is substantially located on the surface of the nanoparticle.

3. The composition of claim 1, wherein the therapeutic agent is a therapeutic protein.

4. The composition of claim 3, wherein the therapeutic protein is a growth factor, a neurotrophic factor, an antibody or mixture of antibodies, a protein that binds to VEGF and/or PIGF.

5-18. (canceled)

19. The composition of claim 3, wherein the therapeutic protein is a mixture of a therapeutic protein and a polycationic protein molecule.

20-21. (canceled)

22. The composition of claim 1, wherein the therapeutic agent is a chemotherapeutic compound.

23-24. (canceled)

25. The composition of claim 1, wherein the therapeutic agent is a therapeutic oligonucleotide.

26-29. (canceled)

30. The composition of claim 1, wherein the therapeutic agent is a composition comprising a chemotherapeutic agent and a therapeutic oligonucleotide.

31-33. (canceled)

34. The composition according to claim 1, wherein the α-tocopheryl compound is a pegylated derivative of α-tocopheryl.

35-41. (canceled)

42. The composition according to claim 1, wherein the phospholipid composition comprises two or more phospholipids.

43-66. (canceled)

67. The composition according to claim 1, wherein the phospholipid composition further comprises a second or third phospholipid.

68-93. (canceled)

94. The composition according to claim 1, further comprising an endosomal escaping agent.

95. The composition according to claim 1, wherein the steroid or steroid derivative is a cholesterol ester_{(C≦24)}.

96. (canceled)

97. The composition according to claim 1, wherein composition further comprises an apoliprotein.

98-99. (canceled)

100. The composition according to claim 1, wherein the composition further comprises a cell permeablizing agent.

101.-102. (canceled)

103. The composition according to claim 1, wherein the composition further comprises a targeting agent.

104. (canceled)

105. The composition according to claim 1, wherein the ratio of the phospholipid composition to the steroid or steroid derivative is from about 1:5 to about 15:1.

106-108. (canceled)

109. The composition according to claim 1, wherein the ratio of the phospholipids in the phospholipid composition comprises a phosphatidylcholine to sphingomyelin ratio from about 10:1 to about 1:2.

110-111. (canceled)

112. The composition according to claim 1, wherein the ratio of the phospholipids in the phospholipid composition comprises a phosphatidylcholine to phospholtidylserine ratio from about 25:1 to about 1:1.

113-114. (canceled)

115. The composition according to claim 1, wherein the steroid or steroid derivative comprises 0.5 w/w % to about 12.5 w/w % of the composition.

116-117. (canceled)

118. The composition according to claim 1, wherein the phospholipid composition comprises from about 10 w/w % to about 45 w/w % of the composition.

119-120. (canceled)

121. The composition according to claim 1, wherein the α-tocopheryl compound comprises from about 5 w/w % to about 60 w/w % of the composition.

122-123. (canceled)

124. The composition according to claim 1, wherein the therapeutic agent comprises from about 0.5 w/w % to about 25 w/w %.

125-127. (canceled)

128. The composition according to claim 1, wherein the composition comprises the therapeutic agent and a polycationic molecule in a ratio from about 10:1 to about 1:10.

129-130. (canceled)

131. The composition according to claim 1, wherein the apolipoprotein comprises from about 20 w/w % to about 70 w/w % of the composition.

132-133. (canceled)

134. The composition according to claim 1, wherein the nanoparticle further comprises monogalactosyldiacylglycerol.

135. The composition according to claim 1, wherein the nanoparticle has a particle size from about 100 nm to about 500 nm.

136-139. (canceled)

140. The composition according to claim 1, wherein the polydispersity index is less than 0.3.

141-145. (canceled)

146. A method of preparing a therapeutic agent-loaded nanoparticle comprising:
(a) admixing a composition with an organic solvent and cholesterol, a composition with an organic solvent and a phospholipid composition, a composition with an organic solvent and an α-tocopheryl compound, and a composition with a solvent and a therapeutic agent to form a first reaction mixture;
(b) removing the organic solvent from the first reaction mixture to form a second reaction mixture;
(c) admixing the second reaction mixture to water by using a homogenizer or a sonication probe to form a prototype nanoparticle; and
(d) admixing one or more therapeutic agents with the prototype nanoparticle to form a therapeutic agent-loaded nanoparticle.

147. A method of preparing a therapeutic agent-loaded HDL mimicking nanoparticle comprising:
(a) admixing a composition with an organic solvent and cholesterol, a composition with an organic solvent and a phospholipid composition, and a composition with an organic solvent and an α-tocopheryl compound to form a first reaction mixture;
(b) removing the organic solvent from the first reaction mixture to form a second reaction mixture;
(c) admixing the second reaction mixture to water to form a prototype nanoparticle.
(d) admixing one or more therapeutic agents with the prototype nanoparticle to form a therapeutic agent-loaded nanoparticle; and
(e) admixing the therapeutic agent-loaded nanoparticle with apolipoprotein A-I to form a therapeutic agent-loaded HDL-mimicking nanoparticle.

148-180. (canceled)

181. A composition prepared according to the methods of any one of method of claim 146.

182. A method of treating a disease or disorder in a patient comprising administering to the patient a therapeutically effective amount of a composition according to claim 1.

183-203. (canceled)

204. A method of inducing neuronal growth comprising administering a composition according to claim 1.

205.-207. (canceled)
